(19) United States
(12) Patent Application Publication
(10) Pub. No.: US 2008/0028480 A1
(43) Pub. Date: Jan. 31, 2008

(54) Markers for Salinity Tolerance in Wheat Plants and the Use Thereof in Breeding Programs

(76) Inventors: Megan Paula Lindsay, Australian Capital Territory (AU); Wolfgang Spielmeyer, Australian Capital Territory (AU); Evans Lagudah, Australian Capital Territory (AU); Richard Alexander James, Australian Capital Territory (AU); Rana Ellen Munns, Australian Capital Territory (AU); ShaoBai Huang, Australian Capital Territory (AU)

Correspondence Address: COOPER & DUNHAM, LLP, 1185 AVENUE OF THE AMERICAS, NEW YORK, NY, 10036, US

(21) Appl. No.: 11/629,835

(22) PCT Filed: Jun. 14, 2005
(86) PCT No.: PCT/AU05/00846

§ 371 Date: Jun. 22, 2007

Related U.S. Application Data
(60) Provisional application No. 60/579,496, filed on Jun. 14, 2004.

(51) Int. Cl. A01H 1/00; A01H 5/00; A23L 1/00; C07H 19/00; C12Q 1/68

(52) U.S. Cl. 800/260; 426/615; 435/6; 536/22.1; 800/320.3

(57) ABSTRACT

The present invention relates to markers of a Nax locus of wheat plants, particularly durum wheat, linked to reduced sodium accumulation, as well as enhanced tolerance to saline and/or sodic soils. The present invention also relates to the use of these markers in breeding programs to produce plants with reduced sodium accumulation, as well as enhanced tolerance to saline and/or sodic soils. Furthermore, the invention relates to plants produced by these breeding programs.

### FIELD OF THE INVENTION

The present invention relates to markers of a Nax locus of wheat plants, particularly durum wheat, linked to reduced sodium accumulation, as well as enhanced tolerance to saline and/or sodic soils. The present invention also relates to the use of these markers in breeding programs to produce plants with reduced sodium accumulation, as well as enhanced tolerance to saline and/or sodic soils. Furthermore, the invention relates to plants produced by these breeding programs.

### BACKGROUND OF THE INVENTION

Soil salinity causes significant reductions in plant productivity, and consequent economic losses associated with reduced grain quality and yield of agricultural crops (Pitman and Läuchli 2002). Over 6% of the world's land is affected by either salinity or sodicity. A large proportion of the Australian wheat belt is at risk of salinisation due to rising water tables, and a further and larger part has soils that are sodic, and underlain with subsoil salinity (Rengasamy 2002). This subsoil salinity is formed in semi-arid zones (with annual rainfall less than 450 mm), and is transient in nature as it moves in and out of the root zone according to soil wetting and drying cycles (Rengasamy 2002). Cultivars of durum wheat are more salt sensitive than bread wheat (Gorham et al. 1990; Rawson et al. 1988), and may yield less when grown on saline soils (Francois et al. 1986; Maas and Grieve 1990). The usual high price of durum wheat on the international market can bring a better return to farmers than bread wheat and other crops, so, breeding new cultivars of durum wheat with improved salt tolerance can allow growers more options in dealing with subsoil salinity. Marker assisted selection is potentially the most efficient approach to developing cultivars that can tolerate saline soils.

There are three avenues by which to introduce salt-tolerance into durum wheat: traditional breeding techniques using physiologically-based phenotyping, marker-assisted selection, and through transformation of genes known to improve Na⁺ exclusion or tissue tolerance. To increase salt tolerance of crops in terms of yield increases and associated economic gains, there is great potential for the introduction of salt tolerance traits into durum wheat using marker-assisted selection (Munns et al. 2002). This approach has successfully been used to introduce various agronomic traits into cereals, and overcomes the problems associated with wheat transformation and market acceptance (Koomeef and Stam 2001). Plant breeding using marker-assisted selection has a proven track-record of successfully incorporating a stable trait into the genome of the target species. However, marker development is dependent on accurate phenotyping, requiring a quantitative measure of a specific trait. An understanding of physiological mechanisms is needed to identify such a trait.

Salt tolerance in the Tritiaceae is associated with sodium exclusion, which limits the entry of sodium into the plant and its transport to leaves. Sodium exclusion from the transpiration stream reaching the leaves is controlled at three stages: (1) selectivity of the root cells taking up cations from the soil solution, (2) selectivity in the loading of cations into the xylem vessels in the roots, and (3) removal of sodium from the xylem in the upper part of the roots and the lower part of the shoot (Munns et al. 2002; Tester and Davenport 2003).

Bread wheat (hexaploid) cultivars are able to exclude Na⁺ from the leaves, however, durum wheat (tetraploid) cultivars lack this trait (Dubcovsky et al. 1996). The Kna1 locus on chromosome 4□L of hexaploid wheat is linked to Na⁺ exclusion and K⁺/Na⁺ discrimination, and subsequently, improved salt tolerance (Dvo\check{r}ák et al. 1994; Shah et al. 1987). Hexaploid wheat has three genomes, A, B and D, but tetraploid wheat has only the A and B genomes. A homoeologue of the Kna1 locus has not yet been found on the A or B genomes. Recently, a novel source of Na⁺ exclusion was identified in a durum landrace (Munns et al. 2000). The landrace had very low rates of Na⁺ accumulation in the leaf blade, as low as bread wheat cultivars, and maintained a high rate of K⁺ accumulation, with consequent high K⁺/Na⁺ discrimination. The low-Na⁺ durum landrace had a K⁺/Na⁺ ratio of 17 whereas the durum cultivars Wollaroi, Tamaroi and Langdon had K⁺/Na⁺ ratios of 1.5, 0.7 and 0.4 respectively (Munns et al. 2000). The bread wheat cultivars Janz and Machete had K⁺/Na⁺ ratios of 10 and 8 respectively. The low Na⁺ trait was shown to confer a significant yield advantage at moderate soil salinity (Husain et al. 2003), indicating that this novel germplasm provides the opportunity to improve the salt tolerance of cultivated durum wheat

Methods for selection of Na⁺ excluding individuals in wheat breeding populations are time-consuming and expensive. In our case, the method involves growing plants in pots using a sub-irrigation system to provide a gradual and uniform exposure to NaCl to the plant, and the harvesting of a given leaf for Na⁺ accumulation. Although this screening method is very reproducible, it is labour intensive and requires a controlled environment. It is not possible to screen plants in the field or with large numbers of individual lines using this method. QTL mapping and marker-assisted selection is a technique that has many advantages over phenotypic screening as a selection tool. Marker-assisted selection is non-destructive and can provide information on the genotype of a single plant without exposing the plant to the stress. The technology is capable of handling large numbers of samples. Although developing a QTL map is laborious, the markers identified may prove to be sufficiently robust to use as the sole selection tool for a specific trait in a breeding program. PCR-based molecular markers have the potential to reduce the time, effort and expense often associated with physiological screening. In order to use marker-assisted selection in breeding programs, the markers must be closely linked to the trait, and work across different genetic backgrounds.

There is a need for further markers which can be used in wheat plant breeding programs, particularly for use in durum wheat breeding programs, to produce plants with reduced sodium accumulation, as well as enhanced tolerance to saline and/or sodic soils.

### SUMMARY OF THE INVENTION

The present inventors have identified molecular markers which allow screening of plants for alleles that confer upon the plant reduced sodium accumulation which is associated with enhanced tolerance to saline and/or sodic soils.

Thus, in one aspect, the present provides a method of identifying a wheat plant with enhanced tolerance to saline and/or sodic soils, the method comprising detecting a nucleic acid molecule of the plant, wherein the nucleic acid molecule is linked to a Nax locus of wheat that comprises an allele that confers enhanced tolerance to saline and/or sodic soils.

In another aspect, the present invention provides a method of identifying a wheat plant having a phenotype of reduced sodium accumulation in an aerial part of the plant, the method comprising detecting a nucleic acid molecule of the plant, wherein the nucleic acid molecule is linked to a Nax locus of wheat that comprises an allele that confers reduced sodium accumulation.

As the skilled addressee will appreciate, the methods can be performed on any nucleic acid of the plant which is a suitable marker for (is linked to) the desired trait. Typically whole genomic DNA, or fractions thereof (for example cleaved with restriction endonucleases), from the plant will be analysed. However, in some instances mRNA or cDNA produced therefrom may be analysed. In a preferred embodiment, the Nax locus is Nax1.

Any technique known in the art for detecting a nucleic acid molecule of interest can be used in the methods of the invention. In one embodiment, the methods comprise:

i) hybridising a second nucleic acid molecule to said nucleic acid molecule which is obtained from said plant,

ii) optionally hybridising at least one other nucleic acid molecule to said nucleic acid molecule which is obtained from said plant; and

iii) detecting a product of said hybridising step(s) or the absence of a product from said hybridising step(s).

Preferably, the second nucleic acid molecule is used as a primer to reverse transcribe or replicate at least a portion of the nucleic acid molecule which is obtained from the plant, or as a hybridisation probe.

In another embodiment, the nucleic acid is detected using a technique selected from the group consisting of: restriction fragment length polymorphism analysis, amplification fragment length polymorphism analysis, microsatellite amplification and/or nucleic acid sequencing.

In a particularly preferred embodiment, the method comprises nucleic acid amplification.

Numerous specific examples of detecting an Nax allele that confers upon a wheat plant reduced sodium accumulation, or enhanced tolerance to saline and/or sodic soils, are described herein. However, considering the disclosure provided herein, the skilled addressee has been provided with suitable information to readily produce alternate strategies to achieve the same end. Examples of preferred detection methods are provided below. In one embodiment, the Nax locus is Nax1 and the amplification is performed using primers which amplify a polymorphic GA-repeat, wherein the polymorphic GA-repeat can be amplified using the primers ATCGCATGATGCACGTAGAG (SEQ ID NO: 11) and ACATGCATGCCTACCTAATGG (SEQ ID NO: 12). In another embodiment, the Nax locus is Nax1 and the amplification is performed using primers which amplify a polymorphic GA-repeat, wherein the polymorphic GA-repeat can be amplified using the primers ATCGCATGATGCACGTAGAG (SEQ ID NO: 11) and GTGGGGGAGGCCGGCCAC (SEQ ID NO: 15).

In a further embodiment, the Nax locus is Nax1 and the amplification is performed using a primer comprising the sequence ATCGCATGATGCACGTAGAG (SEQ ID NO: 11), in conjunction with a primer comprising the sequence ACATGCATGCCTACCTAATGG (SEQ ID NO: 12) or GTGGGGGAGGCCGGCCAC (SEQ ID NO: 15), or at least one primer which is a variant of any one of said primers.

In yet another embodiment, the Nax locus is Nax1 and the amplification is performed using primers which amplify a polymorphic repeat, wherein the polymorphic repeat can be amplified using the primer pairs selected from:

i) ACATCCACGTTTATGTTGTTG (SEQ ID NO: 13) and TTGGTTGCTCAACGTTTACTT (SEQ ID NO: 14),

ii) TGTGGTGCATCACAGGGCTGTTC (SEQ ID NO:21) and AGCGCTTGCATACTCGTCCGG (SEQ ID NO:22), and

iii) AGCAATGAGGATGGTGCTTTCTC (SEQ ID NO:23) and TGTGAGCGACTCCTCGATTTCAG (SEQ ID NO:24).

In a further embodiment, the Nax locus is Nax1 and the amplification is performed using primers pairs comprising the sequences selected from:

i) ACATCCACGTTTATGTTGTTG (SEQ ID NO: 13) and TTGGTTGCTCAACGTTTACTT (SEQ ID NO: 14), or at least one primer which is a variant of any one of said primers,

ii) TGTGGTGCATCACAGGGCTGTTC (SEQ ID NO:21) and AGCGCTTGCATACTCGTCCGG (SEQ ID NO:22), or at least one primer which is a variant of any one of said primers, and

iii) AGCAATGAGGATGGTGCTTTCTC (SEQ ID NO:23) and TGTGAGCGACTCCTCGATTTCAG (SEQ ID NO:24) or at least one primer which is a variant of any one of said primers.

The molecular markers of the invention can be used in wheat breeding programs to select progeny plants which possess an allele of an Nax locus that confer upon the progeny plants reduced sodium accumulation, and/or enhanced tolerance to saline and/or sodic soils.

Thus, in another aspect the present invention provides a method of selecting a wheat plant from a population of wheat plants, the method comprising;

i) crossing two wheat plants of which at least one plant comprises a Nax locus comprising an allele which confers enhanced tolerance to saline and/or sodic soils, and

ii) screening progeny plants from the cross for the presence or absence of said Nax locus by a method of the invention,

wherein progeny with said allele have enhanced tolerance to saline and/or sodic soils when compared to progeny lacking said allele.

A further aspect provides a method of selecting a wheat plant from a population of wheat plants, the method comprising;

i) crossing two wheat plants of which at least one plant comprises a Nax locus comprising an allele which confers reduced sodium accumulation in an aerial part of the plant, and

ii) screening progeny plants from the cross for the presence or absence of said Nax locus by a method of the invention,

wherein progeny with said allele have reduced sodium accumulation when compared to progeny lacking said allele.

Preferably, the method further comprises analysing the plant for other genetic markers.

In one embodiment, the wheat is tetraploid wheat.

In another embodiment, the tetraploid wheat is durum wheat.

In a further embodiment, at least one of the wheat plants of step i) is a hexaploid wheat plant.

In yet another embodiment, the cross is between a durum wheat plant comprising said allele and a hexaploid wheat plant lacking said allele.

Preferably, one of the wheat plants is Line 149, Line 150, Line 151 or a progenitor or progeny plant thereof comprising said allele.

Of the wheat plants which have thus far been identified by the inventors to possess an allele of an Nax locus that confer upon the progeny plant reduced sodium accumulation, and/or enhanced tolerance to saline and/or sodic soils, none are really suitable for cultivation in a commercial setting to be used for producing food (for example flour) or non-food products. Thus, it is particularly desirable to introduce the allele of an Nax locus that confer upon the progeny plant reduced sodium accumulation, and/or enhanced tolerance to saline and/or sodic soils, into the genome of commercially important wheat cultivars whilst maintaining the positive attributes of these plants.

Thus, in a further aspect, the present invention provides a method of introducing a Nax locus into the genome of a wheat plant lacking said locus, the method comprising;

i) crossing a first parent wheat plant with a second parent wheat plant, wherein the second plant comprises a Nax locus which comprises an allele which confers enhanced tolerance to saline and/or sodic soils, and

ii) backcrossing the progeny of the cross of step i) with plants of the same genotype as the first parent plant for a sufficient number of times to produce a plant with a majority of the genotype of the first parent but comprising said allele, wherein progeny plants are screened for the presence or absence of said allele by a method of the invention.

In another aspect, the present invention provides a method of introducing a Nax locus into the genome of a wheat plant lacking said locus, the method comprising;

i) crossing a first parent wheat plant with a second parent wheat plant, wherein the second plant comprises a Nax locus which comprises an allele which confers reduced sodium accumulation in an aerial part of a wheat plant, and

ii) backcrossing the progeny of the cross of step i) with plants of the same genotype as the first parent plant for a sufficient number of times to produce a plant with a majority of the genotype of the first parent but comprising said allele,

wherein progeny plants are screened for the presence or absence of said allele by a method of the invention.

In one embodiment, the first and/or second parent wheat plant is a durum wheat plant.

In another embodiment, the first parent wheat plant is a hexaploid wheat plant.

In a further embodiment, one of the wheat plants is Line 149, Line 150, Line 151 or a progenitor or progeny plant of any one of these comprising said allele.

Also provided is a wheat plant produced using a method of the invention.

In a further aspect, the present invention provides a hexaploid wheat plant identified by a method according to the invention.

In another aspect, the present invention provides a seed of a wheat plant of the invention.

In yet a further aspect, the present invention provides a hexaploid wheat plant comprising an allele of the Nax1 gene on chromosome 2AL which confers enhanced tolerance to saline and/or sodic soils, and/or reduced sodium accumulation in an aerial part of the plant.

In yet a further aspect, the present invention provides a product produced from a wheat plant according to the invention.

Also provided is a product produced from a seed of the invention and/or the plant of the invention. Such products can be food or non-food products. Methods of producing such products are well known to those skilled in the art.

Examples of food products include, but are not limited to, flour, starch, leavened or unleavened breads, pasta, noodles, animal fodder, breakfast cereals, snack foods, cakes, pastries and foods containing flour-based sauces.

Examples of non-food products include, but are not limited to, films, coatings, adhesives, building materials and packaging materials.

The present invention also provides an oligonucleotide capable of being used in methods of the invention, wherein the oligonucleotide is not selected from the group consisting of: ATCGCATGATGCACGTAGAG (SEQ ID NO: 11), ACATGCATGCCTACCTAATGG (SEQ ID NO: 12), ACATCCACGTTTATGTTGTTG (SEQ ID NO: 13) TTGGTTGCTCAACGTTTACTT (SEQ ID NO: 14), CAAATGGATCGAGAAAGGGA (SEQ ID NO: 9) and CTGCCATTTTTCTGGATCTACC (SEQ ID NO: 10).

Preferably, the oligonucleotide is capable of being used as a primer for nucleic replication or reverse transcription.

Preferably, the oligonucleotide comprises the sequence GTGGGGGAGGCCGGCCAC (SEQ ID NO: 15), TGTGGTGCATCACAGGGCTGTTC (SEQ ID NO:21), AGCGCTTGCATACTCGTCCGG (SEQ ID NO:22), AGCAATGAGGATGGTGCTTTCTC (SEQ ID NO:23) or TGTGAGCGACTCCTCGATTTCAG (SEQ ID NO:24), or a variant of any one thereof.

Furthermore, the present invention provides a kit for identifying a wheat plant with enhanced tolerance to saline and/or sodic soils, and/or identifying a wheat plant having a phenotype of reduced sodium accumulation in an aerial part of the plant, the kit comprising at least one oligonucleotide capable of being used in a method of the invention.

The kit may also comprise other components useful for performing the methods of the invention such as, but not limited to, a polymerase for use in DNA amplification procedures.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. Relationship between salinity tolerance and leaf Na⁺ concentration in (A) subspecies durum selections (●) and (B) other tetraploid subspecies selections. Na⁺ concentrations were measured on leaf 3 after 10 d growth in 150 mM NaCl and biomass after 24 d growth in control and salt treatments. All values are means (n=5). Fitted linear regressions are (A) y=−1.84E-04x+0.74 (r²=0.74) and (B) y=−1.1E-04x+0.47 (r²=0.08).

FIG. 2. Relationship of salt tolerance with leaf Na⁺ concentration in families from a cross between subspecies durum selections, Lines 141 and 149. Na⁺ concentrations were measured on leaf 5 after 20 d in 150 mM NaCl and biomass after 24 d. Methods as described in Munns and James (2003). Open circle is the data for Line 149.

FIG. 3. Effect of different levels of salinity on chlorophyll content in leaf 6 of low sodium (●) and high sodium (▪) genotypes grown in the presence of (a) 1 mM NaCl, (b) 75 mM NaCl, and (c) 150 mM NaCl. Leaf 6 emerged 21 days after the salt treatment started. Bars show the s.e.m.

FIG. 4. Effect of three different salinity levels (1, 75 and 150 mM NaCl) on grain yield (g per plant) of low and high sodium genotypes at grain maturity (133 d in salt). Asterisks indicate significant differences from the control and from each other at P=0.05.

FIG. 5. Frequency distribution for leaf Na⁺ concentration (blade of leaf 3, 10 days after its emergence) among 100 F₂ individuals from a cross between Line 149 (P₁) and Tamaroi (P₂) grown at 150 mM NaCl. Data from Munns et al. (2003). Data for parents is the mean ±s.e. (n=10).

FIG. 6. Genetic linkage map on chromosome 2A. One of the linkage groups containing chromosome 2A markers and a QTL for the Na⁺ exclusion trait. The map distances are in centimorgans (cM) as determined using the Kosambi function. Numbers in brackets are LOD scores. The arrow points to the centromere location and the markers shown are on the 2AL region.

FIG. 7. Variation among parents and F₂ progeny in DNA fragments amplified by PCR reaction using microsatellite gwm312. Upper band is the A allele from Tamaroi, lower band the B allele from Line 149. The lanes show the results from Line 149 (lane 1), Tamaroi (lane 2), with seven F₂ progeny, which are either homozygous for B (lanes 3 and 4), for A (lanes 5 and 6) or heterozygous (lanes 7-9).

FIG. 8. Validation of microsatellite gwm312 linked to the Na⁺ exclusion trait on chromosome 2AL on individuals selected with lowest and highest Na⁺, in two populations with different genetic background, each with Line 149 as the parent (P₁) with the Na⁺ exclusion trait. Data for parents is the mean ±s.e. (n=10). In the scoring of the loci on the 15 individuals; A: homozygous parental allele of Tamaroi; B: homozygous parental allele of Line 149 and; H: heterozygous state. The edges of the box closest and furthest to the x axis indicates the 25^{th} and 75^{th} percentile, respectively. Whiskers on the box indicate the 10^{th} and 90^{th} percentile. The line within the box indicates the median.

FIG. 9. Validation of microsatellite gwm312 as a robust marker for the Na⁺ exclusion trait on chromosome 2AL in three populations resulting from backcrosses between Line 149 (B allele) and three different recurrent parents (A allele). 25 individuals were screened at random. The edges of the box indicate the 25^{th} and 75^{th} percentile, respectively. Whiskers on the box indicate the 10^{th} and 90^{th} percentile, while points outside that range are graphed separately. The line within the box indicates the median.

FIG. 10. (A) Line 149 (cultivar of Triticum turgidum ssp. durum) amplicon (SEQ ID NO:1) using gwm312 primers. (B) Westonia (cultivar of T. aestivum) amplicon (SEQ ID NO:2) using gwm312 primers.

FIG. 11. Frequency distribution of plants in a BC₅F₂ population according to leaf 3 Na⁺ concentrations. The frequencies of plants for leaf 3 Na⁺ concentrations for the parental lines Line 149 (grey bars) and Tamaroi (black bars) are shown. The frequencies for homozygous lines for the Line 149 allele are shown as hatched bars, for homozygotes for the Tamaroi allele as reverse-hatched bars, and for heterozygotes as cross-hatched bars.

FIG. 12. Schematic genetic linkage map of chromosome 2AL of durum wheat showing the map location of Nax as a QTL (“Previous Durum 2AL”, see Example 2) and as a single locus (“New Durum 2AL”) with respect to microsatellite markers (gwm, wmc) and wheat EST markers TaA, TaC, TaQ and TaP. The physical map of the syntenic region of rice chromosome 4L with corresponding rice genes is shown on the left.

FIG. 13. Schematic diagram showing mapping of microsatellite markers (gwm, wmc) and wheat EST markers (Ta) to defined physical telomeric deletion bins of chromosome 2AL in Chinese Spring wheat. The percentages describe the approximate percentage of 2AL chromosome arm present in a particular deletion line.

### KEY TO THE SEQUENCE LISTING

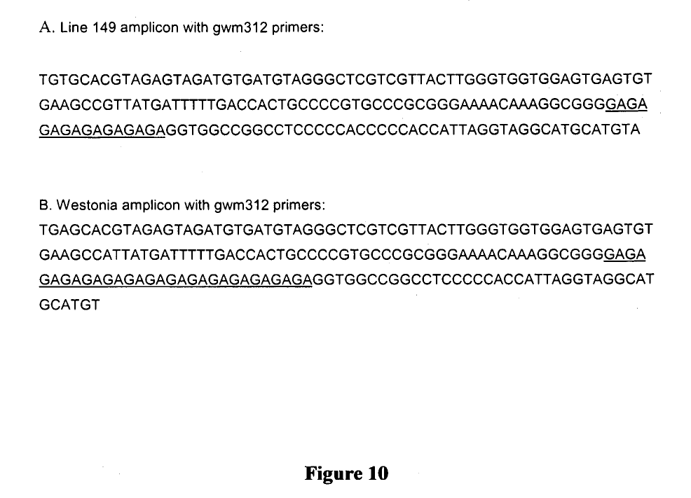

SEQ ID NO:1—Amplicon from Line 149 using gwm312 primers (FIG. 10).

SEQ ID NO:2—Amplicon from Westonia cultivar using gwm312 primers (FIG. 10).

SEQ ID NO's:3 to 24—Oligonucleotide primers.

### DETAILED DESCRIPTION OF THE INVENTION

General Techniques

Unless specifically defined otherwise, all technical and scientific terms used herein shall be taken to have the same meaning as commonly understood by one of ordinary skill in the art (e.g., in cell culture, molecular genetics, wheat breeding, protein chemistry, and biochemistry).

Unless otherwise indicated, the, genetic marker analysis, recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

Selected Definitions

A Nax locus comprises alleles within a population of wheat plants which confer reduced sodium accumulation and/or enhanced tolerance to saline and/or sodic soils upon a wheat plant when compared to a wheat plant lacking one of said alleles. Preferably, the Nax locus is Nax1. It is preferred that the Nax locus is present on the A or B genomes of wheat. The Nax loci described herein do not include and are distinct from the KnaI locus on chromosome 4D of hexaploid wheat (Gorham et al. 1997).

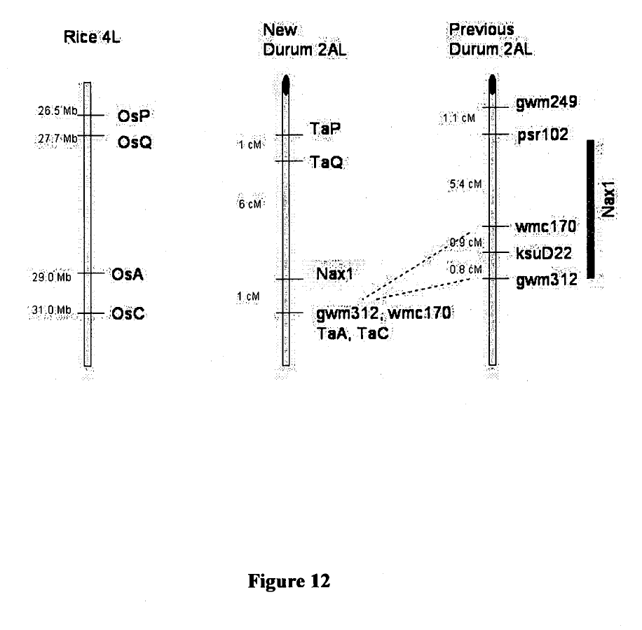

As used herein, the term “Nax1” refers to a region (locus) on the long arm of chromosome 2 of the genome of a wheat plant (see FIG. 12). An allelic variant (allele) of the Nax1 locus has been shown herein to be linked to enhanced tolerance to saline and sodic soils as well as reduced sodium accumulation. Examples of markers of this region, or genetically linked thereto, include AFLP markers AFLP42-1 and AFLP27-1; RFLP markers Xspr102, XksuE16 and XksuD22, as well as microsatellite markers Xgwm249, Xgwm817, TaA, TaC, Xgwm312 (also referred to herein as gwm312) and Xwmc170 (also referred to herein as wmc170) (see the Examples section for further details). Particularly preferred markers of alleles of the Nax1 locus linked to enhanced tolerance to saline and/or sodic soils, as well as reduced sodium accumulation, are Xgwm312, Xgwm817 and Xwmc170.

As used herein, the term “wheat” refers to any species of the Genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. Tetraploid wheat includes T. durum (also referred to herein as durum wheat or Triticum turgidum ssp. durum), T. dicoccoides, T. dicoccum, T. polonicum, and interspecies cross thereof. In addition, the term “wheat” includes potential progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. Particularly preferred progenitors are those of the A genome, even more preferably the A genome progenitor is T. monococcum. A wheat cultivar for use in the present invention may belong to, but is not limited to, any of the above-listed species. Also encompassed are plants that are produced by conventional techniques using Triticum sp. as a parent in a sexual cross with a non-Triticum species (such as rye [Secale cereale]), including but not limited to Triticale.

As used herein, the phrase “enhanced tolerance to saline and/or sodic soils” is considered as relative term. A saline soil is defined as having a high concentration of soluble salts, high enough to affect plant growth. Salt concentration in a soil is measured in terms of its electrical conductivity. As used herein a saline soil has an EC, of at least 1 dS/m, more preferably at least 2 dS/m, more preferably at least 3 dS/m, and even more preferably at least 4 dS/m. ECe is the electrical conductivity of the ‘saturated paste extract’, that is, of the solution extracted from a soil sample after being mixed with sufficient water to produce a saturated paste. Sodic soils have a low concentration of soluble salts, but a high percent of exchangeable Na⁺; that is, Na⁺ forms a high percent of all cations bound to the negative charges on the clay particles that make up the soil complex. Sodicity is defined in terms of the threshold ESP (exchangable sodium percentage) that causes degradation of soil structure. As used herein a sodic soil has an ESP greater than 5, more preferably an ESP greater than 7, more preferably an ESP greater than 9, more preferably an ESP greater than 11, more preferably an ESP greater than 13, and even more preferably an ESP greater than 15. A wheat plant with enhanced tolerance to saline and/or sodic soils is defined as a wheat plant which comprises an allele of a Nax locus linked to this trait, where the presence of the allele means that the plant is more capable of growing, and/or reproducing, in saline and/or sodic conditions when compared to a plant with the same, or similar (such as members of the same species, more preferably members of the same subspecies), genotype but lacking said allele. Indicators of enhanced tolerance to saline and/or sodic soils linked to loci of the invention include, but are not limited to, reduced sodium uptake and/or lower levels of sodium in seeds (whether grown in saline and/or sodic soils or not).

As used herein, the term “reduced sodium accumulation” is considered a relative term. More specifically, the present inventors have identified markers of wheat plants linked to a low rate of Na⁺ accumulation in, for example, the leaf blade. A wheat plant with “reduced sodium accumulation” is defined as a wheat plant which comprises an allele of a Nax locus linked to this trait, where the presence of the allele means that the plant accumulates less sodium in an aerial part of the plant (any above ground part of the plant such as, for example, the stem, leaves and/or seed) when compared to a plant with the same, or similar (such as members of the same species, more preferably members of the same subspecies), genotype but lacking said allele. “Reduced sodium accumulation” can be determined using any method known in the art, such as those described in the Examples.

As used herein, the term “linked” refers to a marker locus and a second locus being sufficiently close on a chromosome that they will be inherited together in more than 50% of meioses, e.g., not randomly. This definition includes the situation where the marker locus and second locus form part of the same gene. Furthermore, this definition includes the situation where the marker locus comprises a polymorphism that is responsible for the trait of interest (in other words the marker locus is directly “linked” to the phenotype). The term “genetically linked” as used herein is narrower, only used in relation to where a marker locus and a second locus being sufficiently close on a chromosome that they will be inherited together in more than 50% of meioses. Thus, the percent of recombination observed between the loci per generation (centimorgans (cM)), will be less than 50. In particular embodiments of the invention, genetically linked loci may be 45, 35, 25, 15, 10, 5, 4, 3, 2, or 1 or less cM apart on a chromosome. Preferably, the markers are less than 5 cM apart and most preferably about 0 cM apart.

An aspect of the invention relates to a method of introducing a Nax allele which confers enhanced tolerance of saline and/or sodic soils and/or reduced sodium accumulation into the genome of a wheat species lacking said allele. The aim of this aspect is to produce a plant with a majority of the genotype of the first parent but comprising said allele. As used in this context, the term “majority” means that the product of the breeding comprises greater than 50%, more preferably at least 60%, more preferably at least 70%, more preferably at least 80%, more preferably at least 90%, more preferably at least 95%, more preferably at least 96%, more preferably at least 97%, more preferably at least 98%, and even more preferably at least 99% of the genome of the first parent.

An allele refers to one specific form of a genetic sequence (such as a gene) within a cell, an individual plant or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”.

As used herein, the term “gene” is to be taken in its broadest context and includes the deoxyribonucleotide sequences comprising the protein coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences; these sequences. The term “gene” encompasses both cDNA and genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

A “polymorphism” as used herein denotes a variation in the nucleotide sequence between alleles of the loci of the invention, of different species, cultivars, strains or individuals of a plant. A “polymorphic position” is a preselected nucleotide position within the sequence of the gene. In some cases, genetic polymorphisms are reflected by an amino acid sequence variation, and thus a polymorphic position can result in location of a polymorphism in the amino acid sequence at a predetermined position in the sequence of a polypeptide. Typical polymorphisms are deletions, insertions or substitutions. These can involve a single nucleotide (single nucleotide polymorphism or SNP) or two or more nucleotides.

As used herein, the “other genetic markers” may be any molecules which are linked to a desired trait of wheat. Thus, the invention also provides for the use of the molecular markers linked to the loci comprising Nax alleles in combination with the use of other markers linked to desirable genes in wheat breeding. Such markers are well known to those skilled in the art and include molecular markers linked to genes determining traits such disease resistance, yield, plant morphology, grain quality, flour colour and the like. Examples of such genes are stem-rust resistance genes Sr2 or Sr38, the stripe rust resistance genes Yr10 or Yr17, the nematode resistance genes such as Cre1 and Cre3, alleles at glutenin loci that determine dough strength such as Ax, Bx, Dx, Ay, By and Dy alleles, the Rht genes that determine a semi-dwarf growth habit and therefore lodging resistance (Eagles et al., 2001; Langridge et al., 2001; Sharp et al., 2001).

Marker Assisted Selection and Detection of a Nax Locus

Marker Assisted Selection and Detection of a Nax Locus Marker assisted selection is a well recognised method of selecting for heterozygous plants required when backcrossing with a recurrent parent in a classical breeding program. The population of plants in each backcross generation will be heterozygous for the gene of interest, for example Nax1 alleles linked to enhanced salinity/sodicity tolerance and/or reduced sodium accumulation, normally present in a 1:1 ratio in a backcross population, and the molecular marker can be used to distinguish the two alleles. By extracting DNA from, for example, young shoots and testing with a specific marker for the introgressed desirable trait, early selection of plants for further backcrossing is made whilst energy and resources are concentrated on fewer plants.

For example, the Nax1 locus of Line 149 of durum wheat is introgressed into an agronomically favourable cultivar of durum wheat (Tamaroi). To further speed up the backcrossing program, the embryo from immature seeds (25 days post anthesis) may be excised and grown up on nutrient media under sterile conditions, rather than allowing full seed maturity. This process, termed “embryo rescue”, used in combination with DNA extraction at the three leaf stage and analysis with markers linked to the Nax1 locus, allows rapid selection of plants carrying the desired trait, which may be nurtured to maturity in the greenhouse or field for subsequent further backcrossing to the recurrent parent.

Any molecular biological technique known in the art which is capable of detecting alleles of a Nax locus linked to enhanced salinity/sodicity tolerance and/or reduced sodium accumulation can be used in the methods of the present invention. Such methods include, but are not limited to, the use of nucleic acid amplification, nucleic acid sequencing, nucleic acid hybridization with suitably labeled probes, single-strand conformational analysis (SSCA), denaturing gradient gel electrophoresis (DGGE), heteroduplex analysis (HET), chemical cleavage analysis (CCM), catalytic nucleic acid cleavage or a combination thereof (see, for example, Lemieux, 2000; Langridge et al., 2001). The invention also includes the use of molecular marker techniques to detect polymorphisms linked to alleles of a Nax locus which confer enhanced salinity/sodicity tolerance and/or reduced sodium accumulation. Such methods include the detection or analysis of restriction fragment length polymorphisms (RFLP), RAPD, amplified fragment length polymorphisms (AFLP) and microsatellite (simple sequence repeat, SSR) polymorphisms. The closely linked markers can be obtained readily by methods well known in the art, such as Bulked Segregant Analysis, as reviewed by Langridge et al. (2001).

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (Ed. M. J. McPherson and S. G Moller (2000) BIOS Scientific Publishers Ltd, Oxford). PCR can be performed on cDNA obtained from reverse transcribing mRNA isolated from plant cells expressing a gene from a Nax locus. However, it will generally be easier if PCR is performed on genomic DNA isolated from a plant.

A primer is an oligonucleotide sequence that is capable of hybridising in a sequence specific fashion to the target sequence and being extended during the PCR. Examples of such primers useful for the methods of the invention include those used for markers Xgwm312, Xgwm817 and Xwmc170 described herein. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can, for example, result in the induction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al. (supra) and Sambrook et al. (supra). Sequencing can be carried out by any suitable method, for example, dideoxy sequencing, chemical sequencing or variations thereof. Direct sequencing has the advantage of determining variation in any base pair of a particular sequence.

Hybridization based detection systems include, but are not limited to, the TaqMan assay and molecular beacons. The TaqMan assay (U.S. Pat. No. 5,962,233) uses allele specific (ASO) probes with a donor dye on one end and an acceptor dye on the other end such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during PCR while a probe with a single mismatched base is not cleaved. Cleavage of the probe dissociates the donor dye from the quenching acceptor dye, greatly increasing the donor fluorescence.

An alternative to the TaqMan assay is the molecular beacon assay (U.S. Pat. No. 5,925,517). In the molecular beacon assay, the ASO probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

Nucleic Acids and Oligonucleotides

Oligonucleotides and/or nucleic acids of the invention hybridize to a Nax locus of wheat plants, or a region of the genome of said plant genetically linked thereto, under stringent conditions. The term “stringent hybridization conditions” and the like as used herein refers to parameters with which the art is familiar. Nucleic acid hybridization parameters may be found in references which compile such methods, Sambrook, et al. (supra), and Ausubel, et al. (supra). For example, stringent hybridization conditions, as used herein, can refer to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA). Alternatively, the nucleic acid and/or oligonucleotides (which may also be referred to as “primers” or “probes”) hybridize to the region of the wheat plant genome of interest under conditions used in nucleic acid amplification techniques such as PCR.

Oligonucleotides of the present invention can be RNA, DNA, or derivatives of either. Although the terms nucleic acid and oligonucleotide have overlapping meaning, oligonucleotide are typically relatively short single stranded molecules. The minimum size of such oligonucleotides is the size required for the formation of a stable hybrid between an oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, even more preferably at least 25 nucleotides in length.

Usually, monomers of a nucleic acid or oligonucleotide are linked by phosphodiester bonds or analogs thereof to form oligonucleotides ranging in size from a relatively short monomeric units, e.g., 12-18, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate.

The present invention includes oligonucleotides that can be used as, for example, probes to identify nucleic acid molecules, or primers to produce nucleic acid molecules. Oligonucleotide of the present invention used as a probe are typically conjugated with a label such as a radioisotope, an enzyme, biotin, a fluorescent molecule or a chemiluminescent molecule.

Oligonucleotides of the invention are useful in methods of detecting an allele of a Nax locus linked to enhanced tolerance to saline or sodic soils and/or reduced sodium accumulation. Such methods, for example, employ nucleic acid hybridization and in many instances include oligonucleotide primer extension by a suitable polymerase (as used in PCR).

A variant of an oligonucleotide of the invention includes molecules of varying sizes of, and/or are capable of hybridising to the wheat genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise the target region. In addition, variants may readily be designed which hybridise close (for example, but not limited to, within 50 nucleotides) to the region of wheat genome where the specific oligonucleotides defined herein hybridise. As an example, a variant of ATCGCATGATGCACGTAGAG (SEQ ID NO: 11) is TAGATGGTGATGTAGGGC (SEQ ID NO: 16) which can be used to produce an amplicon from Line 149 which is slightly smaller, but provides the same linkage to the desired trait, as shown in FIG. 10.

### EXAMPLES

### Example 1

### Assessment of Phenotypes of Wheat Varieties Grown Under Saline Conditions

Background

Screening large numbers of genotypes for salinity tolerance in the field is difficult, due to spatial heterogeneity of soil chemical and physical properties, and to seasonal fluctuations in rainfall. Screening techniques that can be carried out under controlled environments have therefore been used. Biomass production at high salinity (up to 250 mM NaCl) has been measured relative to biomass under non-saline conditions (for example, Kingsbury and Epstein, 1984; Martin et al., 1994). A screen by Sayed (1985) of 5,000 wheat lines, based on survival of high salinity, showed considerable genetic diversity amongst hexaploid and tetraploid lines. However, survival may not correlate with performance in the field. Leaf injury as measured by membrane damage (leakage of ions from leaf discs), premature loss of chlorophyll (using a hand-held meter), or damage to the photosynthetic apparatus (using chlorophyll fluorescence) can identify genotypes able to germinate in, or survive, very high salinities (over 200 mM NaCl), but do not discriminate between genotypes in ability to tolerate the low or moderate salinities typical of many saline fields (50-100 mM NaCl).

Other studies have used measurements of leaf or root elongation rate to identify genetic differences in response to moderate salinity. Short term growth experiments have revealed little difference between genotypes that differ in long term biomass production or yield and so longer term biomass production or yield measurements are preferred, for example for at least two weeks to several months (Nicolas et al., 1993; Rivelli et al., 2002; Shah et al., 1987; Weimberg, 1987; Munns et al., 1995). However, long term growth experiments are demanding of labour and resources, even when limited to a relatively few number of genotypes, and so other techniques such as trait-based selection are preferred.

Traits used for screening germplasm for salinity tolerance have included Na⁺ exclusion (Garcia et al. 1995), K⁺/Na⁺ discrimination (Asch et al., 2000) and Cl⁻ exclusion (Rogers and Noble, 1992). We describe below a screening technique based on Na⁺ exclusion, as genetic differences in Na⁺ exclusion are highly correlated with differences in salinity tolerance between tetraploid and hexaploid wheat (Francois et al., 1986; Gorham et al., 1987).

Methods

Growth Conditions and the Gravel-Based Hydroponic Method

Seeds were selected by weight, surface sterilised with 1% hypochlorite for 15 min, and germinated in Petrie dishes for 3 days. Germinated seeds were planted into 6.5×15.8 cm pots containing quartz gravel, one plant per alternate pot, in 90 L plastic moulded trays containing 144 pots. Trays were subirrigated with either saline or non-saline nutrient solution, as described in Munns et al. (1995). This gravel culture was preferred to other forms of hydroponic culture, as roots were supported, each plant was a separate replicate, the frequent subirrigation and drainage avoided hypoxia, and there was no breakage of lateral roots as occurs in unsupported hydroponics when the solution was changed (Miller 1987). The nutrient solution at full strength was Hoagland and Amon solution No 2, containing 4 mM Ca²⁺ and 1 mM P.

Seedlings were watered initially with tap water, then half strength nutrient solution was introduced 2 days after emergence (DAE), and increased to full strength at 3 DAE. Commencing at 4-6 DAE, 25 mM NaCl was added to the irrigation solution twice daily over three days to a final concentration of 150 mM. Supplemental Ca²⁺ was added (as CaCl₂) to bring the total concentration of Ca²⁺ to 10 mM, and the molar ratio of Na⁺:Ca²⁺ to 15:1. Control treatments always had 1 mM NaCl added to the nutrient solution. The pH was measured twice weekly and adjusted as needed to pH 6.0 with HCl. Root temperature was controlled using condensers in the solution reservoirs and monitored every 5 min using thermocouples. Experiments were conducted in a glasshouse with natural light and controlled air temperature. Salinity tolerance was calculated as shoot dry weight as a percentage of control shoot dry weight.

Comparison of Durum Wheat Lines for Sodium Accumulation Phenotypes

The relationship between the Na⁺ exclusion/sodium accumulation trait and salinity tolerance was investigated by growing a range of tetraploid wheat varieties under saline/non-saline conditions in the greenhouse and measuring leaf Na⁺ contents and biomass production. A collection of 21 tetraploid lines representing five subspecies were randomly selected and 10 plants of each grown in control and salt treatments. Leaf 3 of each seedling (n=5 for each genotype) was harvested 10 days after its appearance, coinciding with approximately 10 days of salt treatment. Leaf 3 was chosen for analysis as it was the first leaf to have fully developed after the salt treatment was initiated. Preliminary experiments had indicated that genotypic differences were greatest for leaf 3 after 10 days. However, any subsequent leaf at any stage of development would probably have shown the same genotypic differences and could have been used. Subsequent leaves have lower concentrations than early leaves, but the genetic differences remain the same (Rivelli et al., 2002).

Remaining shoots (n=10) were harvested at 28 days after emergence (DAE, coinciding with 24 days of treatments). Average daily PAR was 43.6 mol m⁻² d⁻¹. Daily glasshouse air temperature ranged from between 32° C. (day) and 20° C. (night).

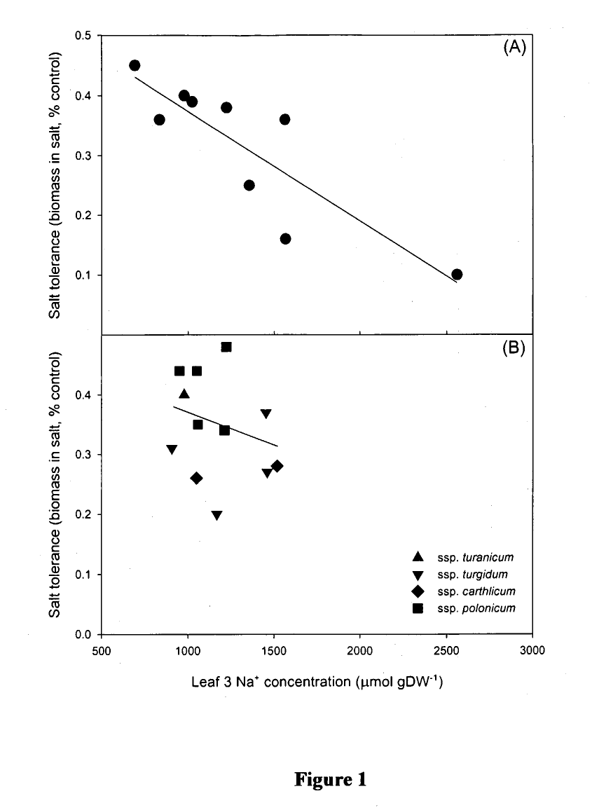

The data (FIG. 1) showed that lower Na⁺ levels correlated with greater salt tolerance (biomass production) across all tetraploids (r²=0.65), but this was dominated by the durum selections with r²=0.74 (FIG. 1A). Na⁺ levels alone did not correlate with salt tolerance in the other tetraploid subspecies. The lower sodium accumulation seen here is presumed to be due to an Na⁺ exclusion mechanism from at least the aerial parts of the plants such as Line 149, and the terms “low sodium accumulation” and “high Na⁺ exclusion” are treated as synonymous. K⁺/Na⁺ ratios correlated well with salt tolerance across all subspecies, but the regression coefficient was not as high as with Na⁺. The correlation of K⁺/Na⁺ with salt tolerance was also driven by the durum selections.

Na⁺ exclusion or K⁺/Na⁺ discrimination did not correlate with salinity tolerance in the non-durum tetraploid genotypes, yet some had high salinity tolerance (FIG. 1B). Polonicums as a subspecies performed well, and the carthlicums and turgidums poorly. This suggests that some of these tetraploids have a greater capacity to handle the salt accumulated in the leaf, the trait of tissue tolerance.

Sodium Accumulation Analysis on Durum Wheat from Segregating Populations

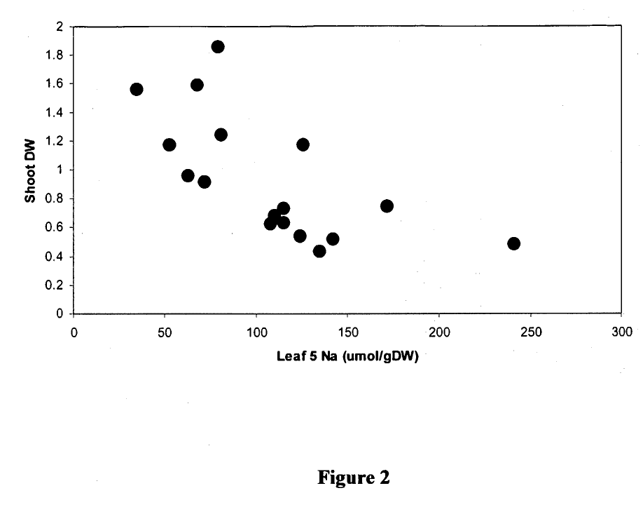

To determine if the association of sodium exclusion and salt tolerance held with a segregating wheat population, plant families derived from a cross between Line 149 (low sodium accumulator) (Australian Winter Cereals Collection, Tamworth, NSW, Australia—Accession No. AUS17045) and Line 141 (high sodium) were assessed for leaf sodium concentration and biomass production. Line 149 was crossed with Line 141 (Munns et al. 2000) and 100 F₂ individuals were selfed to give 100 F₃ families. The F₃ families with the lowest Na⁺ contents were selfed to give F₄ families. These were grown in 150 mM NaCl (15 replicate individuals per family) and shoot growth and leaf Na⁺ were measured at the leaf 5 stage. Salt tolerance, as measured by shoot biomass after 24 d in saline solution, correlated negatively with leaf sodium concentration (FIG. 2). This confirmed the relationship between sodium accumulation/Na⁺ exclusion and biomass production in a common genetic background.

Sodium Exclusion Reduces Leaf Injury and Death Under Saline Growth Conditions

When plants grow in saline soil, some of the salt is taken up by roots and transported to the leaves, where it may increase to toxic high levels. Sodium ion is thought to be the toxic element, not chloride ion. If toxicity occurs, old leaves die, and there are not enough functional leaves to support plant growth at its optimal rate. Plant growth and grain yields are reduced. The relationship of sodium exclusion and leaf injury or death was assessed as follows.

Plants of Line 149 (low sodium accumulator), Line 141 (high sodium accumulator) and four F_{2:4} families from a cross between the two genotypes, two having low leaf sodium concentrations similar to Line 149 and two families having high sodium, were grown in sand culture with three salinity levels, 1, 75 and 150 mM NaCl. These concentrations corresponded to 0.1, 7.5 and 15 dS/m. The area of dead leaf was measured over time. Chlorophyll levels were monitored in main stem leaves at approximately 7-day intervals using a Minolta SPAD 502 meter (Minolta, Osaka, Japan).

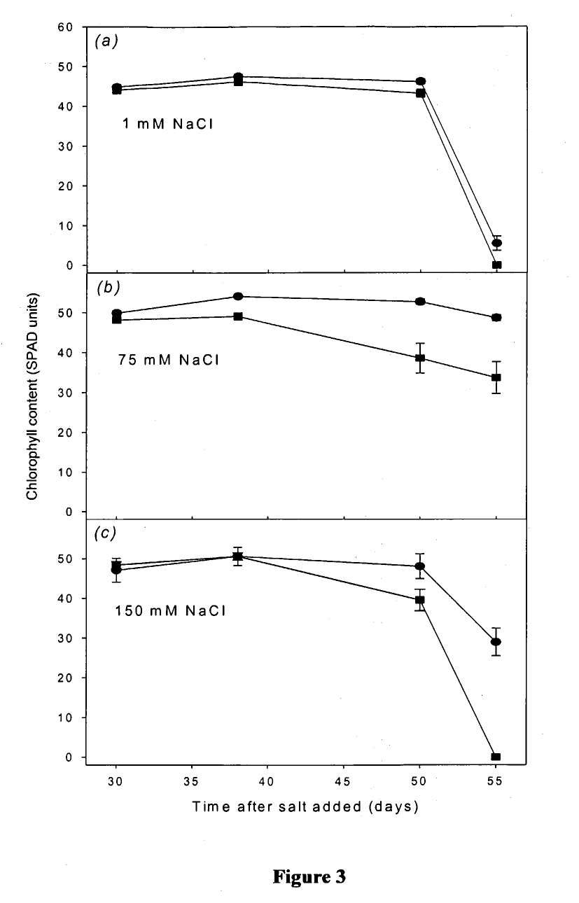

Leaves of the three low sodium lines lived longer than leaves of the three high sodium lines, the start of leaf senescence being prolonged by a week or more in the low sodium lines. For leaf 6, the high sodium lines lost chlorophyll more rapidly and died earlier than the high sodium lines (FIG. 3). Other leaves showed similar results.

Comparison Between Low and High Sodium Families Resulting from 4^{th} Backcross Between Line 149 and cv. Tamaroi

Line 149 and Tamaroi (high sodium accumulator) were crossed and the F1 progeny backcrossed to Tamaroi, followed by selfing, to provide a BC₄F₂ population. These lines were created by growing the F₂ individuals in gravel culture in the presence of 150 mM NaCl, selecting those with the lowest leaf Na⁺ concentration, rescuing them from the saline solution by transplanting into non-saline soil, then performing the backcrossing and selfing. At the BC₄F₁ stage, 350 F₂ seedlings were grown in 150 mM NaCl in gravel culture, and leaf Na⁺ measured after 10 days. One week after this, when leaf 5 was emerging, 28 lines with the lowest sodium and 26 lines with the highest sodium were selected for transplanting into non-saline soil. At this stage, when plants had been exposed to NaCl for a total of 17 d, injury on the first two leaves was assessed.

Twenty-eight BC₄F₂ lines with the lowest sodium and 26 lines with the highest sodium accumulation obtained from this population were assessed for the amount of leaf death of leaf 1 and 2 after two weeks growth. Plants were grown in gravel culture in the presence of 150 mM NaCl as described above.

Table 1 shows the % of leaf death of leaves 1 and 2, assessed visually after two weeks. There was no death of leaf 3, and leaf 4 was only just emerging. The results showed that the sodium exclusion trait minimised leaf injury. The high sodium lines had an average of 77±5% area of dead leaf, whereas the low sodium lines had only 9±3%. Sodium accumulated to 210±10 for the low sodium accumulators, and to 1509±47 for the high sodium accumulators.

These experiments showed that the sodium exclusion/low sodium accumulation trait was associated with reduced leaf injury and death.

TABLE 1
Amount of leaf death of leaves 1 and 2 after two weeks
of plant growth in the presence of 150 mM NaCl.

| BC4F2 selections | | Area of dead leaf (%) |
|---|---|---|
| classification | Number of lines | (mean ± se) |
| Low Na+ | 28 | 9 ± 3 |
| High Na+ | 26 | 77 ± 5 |

Effect of Sodium Exclusion Trait on Yield

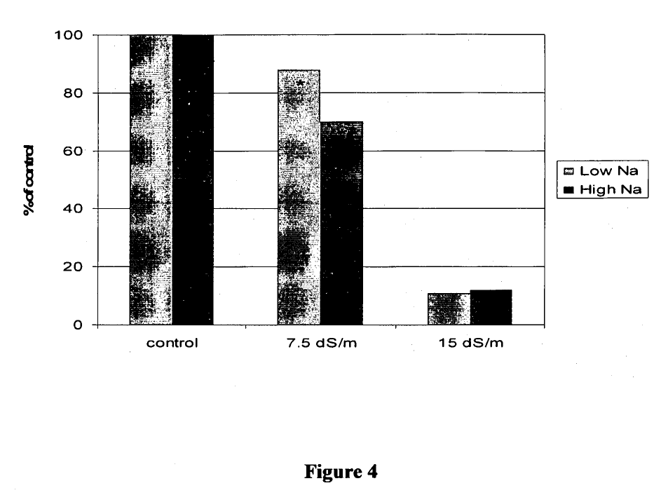

Lines 149 and 141 were used to assess the effects of the sodium exclusion trait on grain yield when plants were grown under saline conditions. Plants were grown in sand culture in a glasshouse with three salinity levels: 1, 75 and 150 mM NaCl, corresponding to 0.1, 7.5 and 15 dS/m. Grain yields at 75 mM NaCl were significantly different between the two genotypes (FIG. 4); the yield of the high Na⁺ genotype was only 70% of control whereas the yield of the low Na⁺ genotype was 88% of the control. This difference was significant at the P=0.05 level. The greater yield of the low Na⁺ genotype was due to enhanced grain number and grain weight in the tiller ears. Grain yields at 150 mM NaCl was equally reduced in both genotypes, being only 12% of controls.

When grown in the field under saline conditions, plants with the low sodium trait (positive for Na⁺ exclusion) yield at least 10% more and in some cases at least 20% more than the corresponding plants without the low sodium trait.

### Example 2

### Mapping of Salinity Tolerance Locus on Chromosome 2AL of Triticum turgidum ssp. durum

Materials and Methods

Plant Material

A mapping population was derived from the cross between Line 149, a low-Na⁺ landrace of Triticum turgidum L. ssp. durum (Desf.) referred to in Munns et al. (2000) as selection number 126-775a, and the Australian durum wheat cultivar, Tamaroi, using 100 F₂ phenotyped individuals. The F_{2:3} progeny were also phenotyped for sodium accumulation (n=15) and the data used to confirm the single-plant data for the F₂ phenotype.

Two other populations were developed to verify the linkage of the marker to the Na⁺ exclusion trait, using crosses of Line 149 to two other high Na⁺ parents with unrelated genetic backgrounds: the cultivar Wollaroi, and the very high Na⁺ landrace Line 141 (Accession No. AUS12818). Populations of 100 F₂ individuals were developed, and the F_{2:3} progeny means (n=15) were used to verify the phenotype of the F₂ individuals.

Two additional populations were developed to test the usefulness of the markers for detecting the Na⁺ exclusion trait in backcrosses with genetically unrelated breeding lines. Lines used were the advanced breeding lines BL960273 and BL961111, the latter now released as the cultivar Bellaroi. Low Na⁺ F₂ individuals from the Tamaroi×Line 149 cross were backcrossed into Tamaroi. BC₃F₂ generation plants were then backcrossed into the breeding lines BL960237 and BL961111, which were self fertilised and backcrossed a second time. BC₃F₂ generation plants were used to verify the usefulness of the marker.

Phenotyping

Plants were grown in gravel culture using an automatic sub-irrigation system as described in Example 1. Pots were sub-irrigated with half-strength Hoagland's solution and 150 mM NaCl, and phosphate was reduced to 50 μM. At 6 d after seedling emergence, when leaf 2 was half-expanded, NaCl salt solution was added to the irrigation solution twice-daily over three days in 25 mM increments to make up the final concentration of 150 mM NaCl. CaCl₂ was added to bring the Ca²⁺ concentration to 8 mM. The electrical conductivity and pH were monitored twice-weekly. For the F₂ measurement of the Tamaroi×Line 149 population, average daily PAR was 17.4 and average glasshouse air temperatures were 21° C. (day) and 20° C. (night). Conditions for measuring other populations were similar.

Na⁺ accumulation in the blade of the third leaf, 10 days after emergence, was measured in all plants according to the method of Munns et al. (2000). Parental lines were replicated ten times. Leaf material was harvested, washed in distilled water, dried at 70° C. for 2 days, extracted in 500 mM HCl at 80° C. for 1 hour, and Na⁺ concentration was measured by atomic absorption spectrometry (Varian Spectra AA-300).

Genotyping

Genomic DNA

Plants that were grown in salt tanks for phenotyping were transplanted into soil and allowed to grow for approximately 4 weeks prior to DNA extraction. Leaf material from plants was harvested and DNA extracted as described by Lagudah et al. (1991). For this Example, DNA was extracted from plants in five populations, as follows: (1) Tamaroi×Line 149 population: 100 F₂ individuals, 60 F₃ families comprising the 30 extremes for Na⁺ concentration (the 15 with highest Na⁺ in the F₂ generation, the 15 with lowest Na⁺) and 30 F₃ families representing the distribution range of Na in the remaining 70 lines. (2) Wollaroi×Line 149 population: 30 extreme F₂ individuals. (3) line 141×Line 149 population: 30 extreme F₂ individuals, and 30 extreme F₃ families. (4) BL961111 backcross population: 25 BC₃F₂ random individuals. (5) BL960273 backcross population: 25 BC₃F₂ random individuals. DNA was pooled from the 15 individual F₂ plants with the lowest or highest Na⁺ concentrations respectively. This constituted the material used for the bulked segregant analysis.

AFLP Markers

AFLP analysis was performed according to the method of Vos et al. (1995) using Pst1 and Mse1 restriction enzymes and adapted primers. The sequences of AFLP adapters and primers are listed in Table 2. The selective primer set (Mse+3 and Pst+3) contained 144 primer combinations. For pre-amplification, Mse1 and Pst1 digested genomic DNA was amplified with Mse+1 and Pst+1 primers to produce a secondary template. Mse+3 and Pst+3 primers were used to selectively amplify AFLP fragments. Pst+3 primers (50 ng) were labeled with ³³P-ATP (10 mCi/μl) using T4 polynucleotide kinase (10 U/ml) and PNK buffer. Samples were incubated at 37° C. for 1 h followed by 70° C. for 10 min to inactivate the kinase.

TABLE 2

Mse and Pst primers and adapters for
AFLP pre-amplification and selective
amplification reactions.

| | |
|---|---|
| Primer/Adapter | Sequence |

| | |
|---|---|
| Mse adapter | (SEQ ID NO's: 3 and 4 respectively) |
| | 5′ GACGATGAGTCCTGAG |
| | TACTCAGGACTCAT5′ |
| Mse | (SEQ ID NO: 5) |
| | 5′ GATGAGTCCTGAGTAAG |
| Mse + 1 | Mse + G |
| Mse + 3 | Mse + GAA |
| | Mse + GAC |
| | Mse + GAG |
| | Mse + GAT |
| | Mse + GCA |
| | Mse + GCC |
| | Mse + GCG |
| | Mse + GCT |
| | Mse + GTA |
| | Mse + GTC |
| | Mse + GTG |
| | Mse + GTT |
| Pst adapter | (SEQ ID NO's: 6 and 7 respectively) |
| | CACGATGGATCCAGTGCA 3′ |
| | 3′ GACGTGCTACCTAGGTC |
| Pst | (SEQ ID NO: 8) |
| | 5′ GATGGATCCAGTGCAGAG |
| Pst + 1 | Pst + A |
| | Pst + T |
| Pst + 3 | Pst + ACA |
| | Pst + ACC |
| | Pst + ACG |
| | Pst + AAG |
| | Pst + AGA |
| | Pst + AGT |
| | Pst + ATC |
| | Pst + ATG |
| | Pst + TGA |
| | Pst + TAC |
| | Pst + TGG |
| | Pst + TCT |

The PCR touchdown cycle was: 94° C./30 s, 65° C./30 s, 72° C./1 min, followed by 12 cycles where the annealing temperature dropped to 57° C. over 12 cycles, followed by 23 cycles with an annealing temperature of 57° C. Selective amplification PCR product (20 μl) and 10 μl of loading dye (98% formamide, 10 mM EDTA pH 8.0, containing bromophenol blue and xylene cyanol as tracking dyes) was denatured at 95° C. for 5 minutes. Denatured sample (3 μl) was loaded onto 6% denaturing gels. At completion of the run, the gel was neutralized in glacial acetic acid/20% methanol solution for 20 min, dried on a glass plate (65° C. for 5 h) and exposed to film (Kodak, Biomax MR). AFLPs linked to Na⁺ exclusion were identified by ‘bulked segregant analysis’, i.e. on the basis of bands being present in one parent and the bulked DNA from the 15 extreme individual F₂ plants relating to that parent, and not in the other parent and the bulked DNA from the 15 extreme individuals relating to it.

Microsatellite Markers

A group of 103 wheat microsatellite markers were used to screen the parental lines, Tamaroi and Line 149 (Table 3). Microsatellites were chosen on the basis of their map location in hexaploid wheat in an attempt to establish complete A- and B-genome coverage. The microsatellites that were polymorphic between the parents were used in mapping. All amplifications were performed in 20 ul aliquots containing 1.5 mM MgCl₂, 200 μM dNTP, 200 μM 1×PCR buffer (Boehringer Mannheim), 2 U Taq DNA polymerase and 100 ng genomic DNA. Genomic DNA was amplified using a step-down PCR program: 95° C./4 min, 15 cycles of 94° C./30 s, 65° C.-50° C./30 s decreasing by 1° C./cycle, 72° C./80 s, 30 cycles of 94° C./15 s, 72° C./45 s, followed by a 4° C. holding step. The PCR products were separated using 1.8% metaphor agarose gel. Microsatellites that produced polymorphisms between the parental lines were used to screen individuals in three different crosses. These were (i) 60 F₂ individuals from the Tamaroi×Line 149 population (including the 30 extreme individuals and the 30 F₂ individuals evenly distributed through the population), (ii) 30 extreme individuals from the Wollaroi×Line 149 population and (ii) 30 extreme individuals from the line 141×Line 149 population.

RFLP Markers

Restriction endonuclease digestion and Southern hybridization were performed according to standard methods (Sambrook et al., supra; Seah et al., 1998). Parental lines were digested with restriction enzymes DraI, EcoRV, EcoRI, HindIII, NcoI, XbaI, BamHI, SacI, BglII, and NdeI, and screened with 20 RFLP markers for chromosome 2, based on published consensus linkage maps from hexaploid wheat. These markers (Xabc305, Xbcd402, Xcdo1376, Xpsr102, Xpsr107, Xpsr109, Xpsr112, Xpsr126, Xpsr131, Xpsr135, Xpsr137, Xpsr143, Xpsr146, Xpsr151, Xpsr609, Xpsr908, Xpsr932, XksuH11, XksuE16 and XksuD22) were selected according to preliminary microsatellite analysis which suggested a linkage to chromosome 2. The last two markers showed strong association with the Na⁺ exclusion trait in the 60 F₂ individuals from the Tamaroi×Line 149 population. These RFLP markers were used to genotype the 30 extreme F₂ individuals from the Wollaroi×Line 149 population, and 30 extreme individuals from the line 141×Line 149 population.

TABLE 3
The microsatellite markers used to map the sodium exclusion
locus to chromosome 2AL (markers that were polymorphic for
Tamaroi and Line 149 are highlighted in bold type).

| 1A | 2A | 3A | 4A | 5A | 6A | 7A |
|---|---|---|---|---|---|---|
| Xgwm099 | Xgwm249 | Xgwm369 | Xgwm160 | Xgwm126 | Xgwm169 | Xgwm060 |
| Xwmc024 | Xgwm275 | Xgwm480 | Xwmc258 | Xgwm156 | Xgwm427 | Xgwm130 |
| Xwmc312 | Xgwm312 | Xwmc011 | Xwmc262 | Xgwm186 | Xgwm494 | Xgwm233 |
| | Xgwm339 | Xwmc050 | Xwmc313 | Xgwm291 | Xgwm570 | Xgwm282 |
| | Xgwm359 | Xwmc169 | | Xgwm293 | Xwmc163 | Xgwm332 |
| | Xgwm372 | Xwmc428 | | Xgwm304 | | Xwmc017 |
| | Xgwm425 | | | Xwmc096 | | Xwmc083 |
| | Xgwm445 | | | Xwmc110 | | Xwmc247 |
| | Xgwm512 | | | Xwmc154 | | Xwmc283 |
| | Xgwm515 | | | | | Xwmc346 |
| | Xgwm614 | | | | | Xwmc405 |
| | Xgwm636 | | | | | |
| | Xwmc170 | | | | | |
| 1B | 2B | 3B | 4B | 5B | 6B | 7B |
| Xgwm153 | Xgwm120 | Xgwm108 | Xgwm107 | Xgwm067 | Xgwm133 | Xgwm043 |
| Xgwm268 | Xgwm148 | Xgwm340 | Xgwm113 | Xgwm371 | Xgwm219 | Xgwm146 |
| Xgwm273 | Xgwm374 | Xgwm376 | Xgwm251 | Xgwm408 | Xgwm361 | Xgwm302 |
| Xgwm498 | Xgwm388 | Xgwm389 | Xgwm495 | Xgwm443 | Xgwm508 | Xgwm333 |
| Xwmc406 | Xgwm429 | Xgwm493 | | Xgwm604 | Xgwm613 | Xgwm344 |
| | Xgwm501 | Xwmc043 | | Xwmc028 | Xgwm626 | Xgwm400 |
| | Xgwm526 | Xwmc236 | | Xwmc149 | | Xgwm537 |
| | Xgwm630 | Xwmc334 | | Xwmc235 | | Xgwm569 |
| | Xwmc035 | Xwmc360 | | | | Xgwm577 |
| | Xwmc339 | | | | | Xwmc402 |

Mapping

A linkage map was created from the genotypic data obtained from the AFLP, microsatellite and RFLP analyses of the 60 F₂ individuals from the Tamaroi×Line 149 population, using 22 AFLP primer combinations, 73 microsatellites and 10 RFLPs, and the phenotypic data from the sodium accumulation assays. Single point linkage analysis (P<0.001) was performed using MapManager QTX version b13 software (Manly et al. 2001).

Results

Phenotyping and Genetics of Na⁺ Exclusion Trait

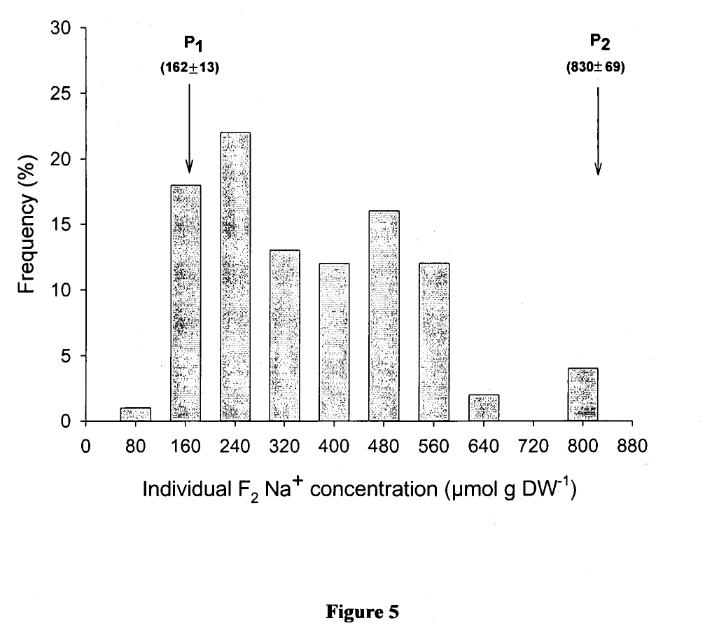

The low Na⁺ parent (Line 149) had an approximately five fold lower Na⁺ concentration than the high Na⁺ parent (Tamaroi). The distribution of sodium levels in F₂ individuals from the cross between them was skewed toward the level in Line 149, indicating that the low Na⁺ trait was dominant (FIG. 5). Genetic analysis indicated that the progeny in the F₂ generation segregated for Na⁺ accumulation in a 15:1 low:high Na⁺ ratio, indicating two genes of major effect, with interacting dominance. Comparison between the F₂ individuals and F₃ progeny demonstrated that the trait had a very high heritability. The realised heritability was 0.90. Differences between the parents and low/high Na⁺ F₂ individuals that were used in bulked segregant analysis are summarized in Table 4. Selective genotyping of the Tamaroi×Line 149 population was performed using bulked segregants based on pooled DNA from 15 low Na⁺ F₂ individuals and 15 high Na⁺. Molecular marker differences between the pair of pooled DNA constituted the basis for identification of linked markers associated with the Na⁺ trait.

Genetic Mapping

The first approach used was an AFLP analysis of the parents, Tamaroi and Line 149, and the bulked F₂ segregants from the cross. Of the 144 primer combinations used, many revealed polymorphisms between the parents and 22 combinations each produced at least 5 polymorphisms. Individual F₂ progeny within the bulked segregants were then tested to validate the association of the putatively linked AFLP markers. One AFLP primer combination (AFLP 42) had a high association with the Na⁺ distribution.

TABLE 4
Na⁺ concentration (μmol g⁻¹ DW) in parental lines and F₂ generation
of the Tamaroi × Line 149 population (low and high Na⁺
selections are the 15 F₂ individuals from the 100 F₂ progeny
with the lowest and highest Na⁺ concentration, respectively,
that were used for the bulked segregant analysis. Parental
means were obtained in the same experiment (10 replicates)).

| Generation | Germplasm | Mean and s.e. | Range |
|---|---|---|---|
| P₁ | Line 149 (n = 10) | 162 ± 13 | 139-270 |
| P₂ | Tamaroi (n = 10) | 830 ± 69 | 621-1244 |
| F₂ | Low Na⁺ selections (15) | 138 ± 4 | 116-165 |
| F₂ | High Na⁺ selections (15) | 643 ± 26 | 551-835 |

The second approach used was a QTL analysis based on 60 F₂ individuals representing the full range of Na⁺ concentrations, and including all lines used in the bulked segregants. This used the 22 AFLP markers identified to be polymorphic between the parents and the bulked segregants, and the microsatellites available for the A and B genomes. Out of 103 microsatellite markers tested, 35 were polymorphic between the parents. This group of markers was used to screen the 60 F₂ individuals.

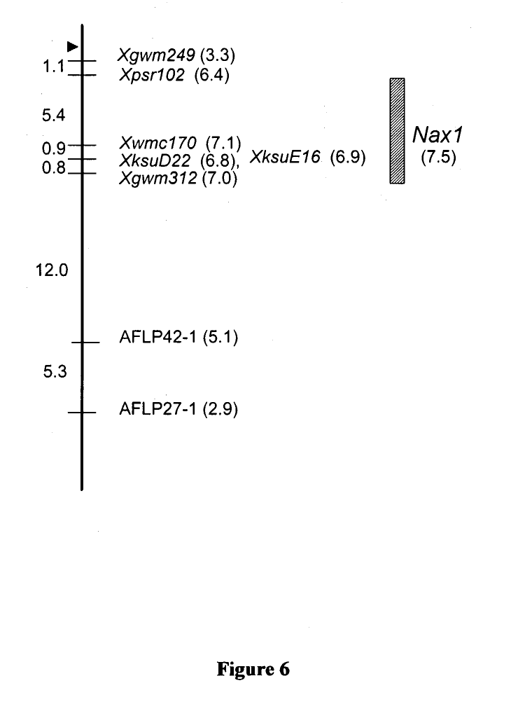

The genotypic data from the microsatellite and AFLP screens were imported into MapManager, and using a high stringency mapping approach (LOD score of 3), several putative linkage groups were identified. Results from interval mapping indicated that a QTL with strong linkage to the Na⁺ exclusion trait was located on chromosome 2AL. The markers that map to this region were tightly linked (FIG. 6). In this initial screen, three microsatellite markers located on chromosome 2AL, Xgwm249 (CAAATGGATCGAGAAAGGGA (SEQ ID NO: 9) and CTGCCATTTTTCTGGATCTACC (SEQ ID NO: 10)), Xgwm312 (ATCGCATGATGCACGTAGAG (SEQ ID NO: 11) and ACATGCATGCCTACCTAATGG (SEQ ID NO: 12)) and Xwmc170 (ACATCCACGTTTATGTTGTTG (SEQ ID NO: 13) TTGGTTGCTCAACGTTTACTT (SEQ ID NO: 14)), were linked to the Na⁺ exclusion/low sodium accumulation trait and had LOD scores of 3.3, 7.0 and 7.1 respectively. Six AFLP markers were linked to the microsatellite markers on chromosome 2AL. The two most tightly linked AFLP markers, AFLP42-1 and AFLP27-1, had LOD scores of 5.1 and 2.9 respectively.

In order to further increase the number of markers on the chromosome 2AL interval associated with the trait, RFLP markers that had previously been mapped to a similar interval in hexaploid wheat were selected. Of the 20 RFLPs from group 2 chromosomes tested, 10 were polymorphic between the parents and were tested on the 60 F₂ segregants. Three RFLP loci, namely Xpsr102, XksuE16 and XksuD22 were tightly linked to the Na⁺ exclusion trait and had LOD scores of 6.4, 6.9 and 6.8 respectively (FIG. 6).

Interval mapping of the Tamaroi×Line 149 population revealed that the QTL located on chromosome 2AL showed significant association with the Na⁺ exclusion trait having a LOD score of 7.5 (FIG. 6). This locus accounts for 38% of the phenotypic variation of the trait. The markers that had the closest association with the Na⁺ exclusion trait were the RFLP markers XksuD22 and XksuE16, and the microsatellites Xgwm312 and Xwmc170. The relative map locations of these four markers are consistent with previous reports of their location in the centromere region of chromosome 2AL. On the long arm of chromosome 2A in hexaploid wheat, Xpsr102 maps to a position 6.0 cM from the centromere, Xgwm312 maps 14.0 cM from the centromere, XksuE16 maps 15.5 cM from the centromere and XksuD22 maps 17.6 cM from the centromere (Roder et al. 1998; www://wheat.pw.usda.gov/). A specific genetic interval for Xwmc170 is not currently available, however public information about this microsatellite is available from the Australian National Wheat Molecular Marker Database (http://www.scu.edu.au). Based on the map location of the most closely linked markers, we predict that the Na⁺ exclusion locus, which we designate Nax1, is located on the long arm of chromosome 2AL, at an approximate position between 6 and 14 cM from the centromere (FIG. 6). The allelic contribution of the QTL located on chromosome 2A is predominantly from the Na⁺ excluding parent.

Marker Validation

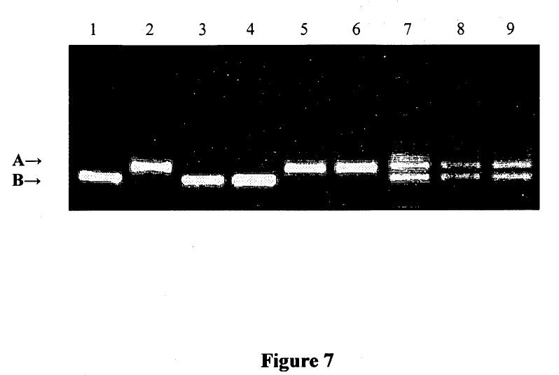

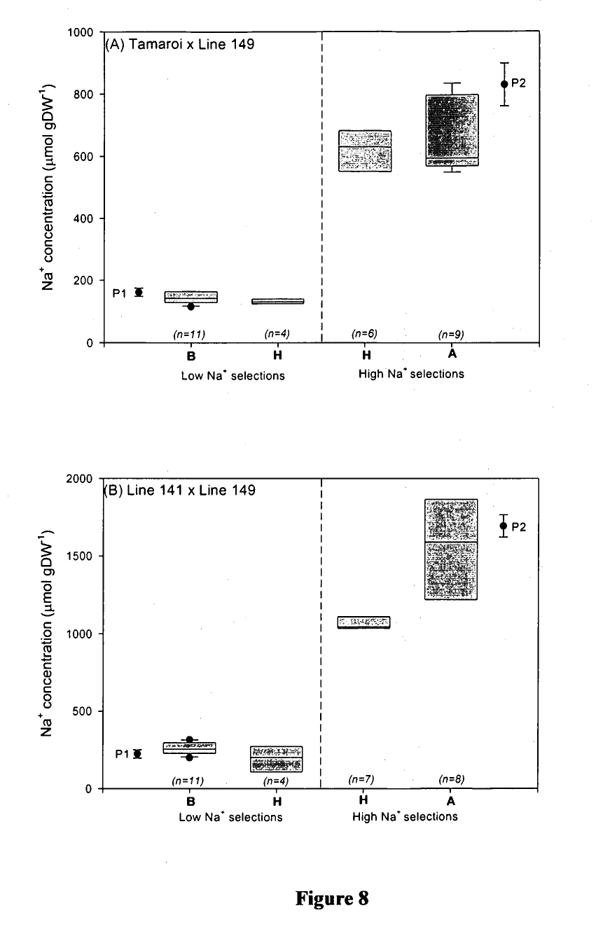

The two co-dominant microsatellite markers that were most closely linked to the Na⁺ exclusion locus on chromosome 2AL from the Tamaroi×Line 149 population were selected to validate whether alleles inherited from Line 149 were associated with the low Na⁺ exclusion trait. These markers are gwm312 (FIG. 7) (Forward: ATCGCATGATGCACGTAGAG (SEQ ID NO: 11) and Reverse: ACATGCATGCCTACCTAATGG (SEQ ID NO: 12) and wmc170 (not shown) Forward: ACATCCACGTTTATGTTGTTG (SEQ ID NO: 13) and Reverse: TTGGTTGCTCAACGTTTACTT (SEQ ID NO: 14)). The markers were first tested on the 30 extreme individuals from the Tamaroi×Line 149 population—the F₂ individuals with the 15 highest and 15 lowest concentrations of leaf Na⁺. There was a strong relationship between the presence of the alleles inherited from the low Na⁺ parent, designated gwm312B (abbreviated as ‘B’), and the low Na⁺ uptake trait (FIG. 8A). Individuals with high Na⁺ uptake were associated with the alternative allele (abbreviated as ‘A’) from the high Na⁺ parent, Tamaroi. The markers also identified several heterozygous F₂ individuals (‘H’) in both low and high Na⁺ uptake groups (FIG. 8A). The microsatellite wmc170 gave identical results as gwm312 (data not shown), because no recombinants between the markers were identified among the extreme subset of low and high Na⁺ uptake F₂ individuals.

The markers were tested on a different population, resulting from a cross between Line 149 and a durum landrace with exceptionally high leaf Na⁺, Line 141. Tamaroi and Line 141 are genetically unrelated (Husain et al. 2003; Munns et al. 2003), therefore, this was a stringent test of the ability of the marker to identify individuals with the Na⁺ exclusion trait in different genetic backgrounds. The phenotype of each of the F₂ individuals was determined using the standard phenotypic screen, and the 30 extreme F₂ individuals were selected for marker validation (FIG. 8B). The results again showed a close linkage with the Na⁺ exclusion trait, in that the low Na⁺ individuals were homozygous B or heterozygous (H), and the high Na⁺ individuals were homozygous A or heterozygous (FIG. 8B). In this subset of extreme F₂ individuals from the 141×149 population, there were recombinants between the two microsatellites. This was evident in that three of the low Na⁺ individuals were homozygous B for gmw312 but heterozygous for wmc170, and that four of the high Na⁺ individuals were homozygous A for gms312 but heterozygous for wmc 170 or vice versa. This suggested that more extensive recombination at meiosis was occurring for the two landraces than for one landrace and a cultivar. However, in neither case were the low Na⁺ individuals homozygous for the allele for the high Na⁺ parent, or vice versa.

Validation for Marker-Assisted Selection in a Breeding Program

To test the usefulness of the microsatellite markers in identifying the Na⁺ exclusion trait in a breeding program, three populations of different genetic background were selected and screened with the microsatellite marker gwm312. These populations resulted from an initial cross between Line 149 and Tamaroi, selection of the lowest Na⁺ F₂ individuals, which were then crossed again with either Tamaroi or two other advanced durum breeding lines, selfed, and this process repeated two more times (see Example 1). These breeding lines had even higher leaf Na⁺ concentrations than did Tamaroi (Table 5). Then, 25 BC₃F₂ individuals from each population were assessed for Na⁺ exclusion using the standard phenotypic screen, and genotyped using gwm312.

TABLE 5
Association of microsatellite gwm312 with Na⁺ exclusion phenotype
in 25 individuals selected at random from three back-crossed populations
with different genetic backgrounds (low Na⁺ selections from a BC₁F₂
family (Tamaroi × Line 149) which were backcrossed twice more with
three recurrent parents: Tamaroi, BL960273 and BL961111, to generate
three BC₃F₂ families with genetically unrelated backgrounds. In the
scoring of the alleles: A is the homozygous parental allele of
Tamaroi, B the homozygous parental allele of Line 149, and H is the
heterozygous state (see FIG. 7)).

| | | Leaf Na⁺ | | |
|---|---|---|---|---|
| | | concentration | t-test | |
| Population/Genotype | Allele | (μmol gDW − 1)¹ | B vs A | B vs H |

(a) BC₃F₂ populations

| | | | | |
|---|---|---|---|---|
| Tamaroi × Line 149 | B | 318 ± 69 | 0.01 | ns² |
| | H | 428 ± 37 | | |
| | A | 650 ± 84 | | |
| BL960273 × Line 149 | B | 391 ± 39 | 0.01 | 0.05 |
| | H | 637 ± 30 | | |
| | A | 1044 ± 62 | | |
| BL961111 × Line 149 | B | 326 ± 40 | 0.01 | 0.01 |
| | H | 647 ± 50 | | |
| | A | 1010 ± 61 | | |

(b) Genotype means ± se

| | | |
|---|---|---|
| Line 149 | B | 169 ± 9 |
| Tamaroi | A | 895 ± 46 |
| BL960273 | A | 1278 ± 39 |
| BL961111 | A | 1245 ± 62 |

¹means ± s.e.
²ns—not significant

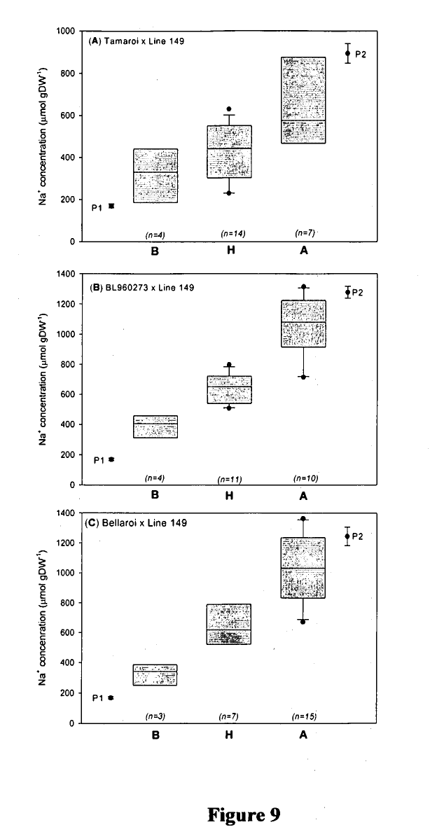

There was high correlation between low Na⁺ concentrations and the presence of the B allele of gwm312 in all three populations (Table 5). All of the lowest Na⁺ individuals in each population were homozygous for the B allele, and all of the highest Na⁺ individuals were homozygous for the A allele (FIG. 9). As was observed in the initial verification of the marker from the Tamaroi×Line 149 cross, several heterozygous individuals were identified in both the high and low Na⁺ classes (FIG. 9). However, the most salt-tolerant individuals carried only the B allele.

These results show that a breeding program based on selection for the B allele will lead to the transfer of the Na⁺ exclusion/low sodium accumulation trait into recurrent parents of very different genetic background.

Discussion

A locus for the Na⁺ exclusion/low sodium accumulation trait in Line 149 was successfully mapped using a QTL approach. Several AFLP, RFLP and microsatellite markers were linked to the gene(s) at the QTL, designated Nax1. The markers that mapped to the Nax1 locus have previously been mapped to chromosome 2A in hexaploid wheat. According to Roder et al. (1998) and Harker et al. (2001), Xgwm312 and Xwmc170, the two microsatellites most closely linked to the locus, map to the long arm of chromosome 2A in hexaploid wheat. Nachit et al. (2001) found markers that map to chromosome 2AL in hexaploid wheat map to a similar interval in tetraploid wheat. The Nax1 locus identified in this study maps to a position between approximately 6.0 and 14.0 cM from the centromere on chromosome 2AL.

The Nax1 locus is not homeologous to the only QTL previously mapped in hexaploid wheat for Na⁺ exclusion and its associated K⁺/Na⁺ discrimination—the Kna1 locus. This has been mapped on chromosome 4DL (Dubcovsky et al. 1996) and is therefore different to Nax1. Other QTLs related to the control of Na⁺ transport have been reported in other species, but have not been mapped with the same precision as the Kna1 or Nax1 loci.

In barley, clusters of QTLs for salt tolerance at the vegetative stage were found on chromosomes on group 1, 4, 6 and 7 (Ellis et al. 1997). A later study by Ellis et al. (2002) with different barley parents reported QTLs for salt tolerance at the vegetative stage on chromosomes 2, 5 and 7. QTLs for shoot and root weight on chromosome 5 had the relatively high LOD score of 6.6 and 7.0 respectively. In field trials, QTLs for grain weight and grain nitrogen with high LOD scores, ranging from 8 to 21, were found on chromosomes 3, 4, 5 and 7. In all, there were 16 primary QTLs whose location had a high level of confidence, but these in total accounted for no more than 35% of the total phenotypic variation. None of these QTLs reported for any species or any cross have been verified in genetically unrelated backgrounds. These results indicate it is not easy to identify reliable loci for salt tolerance or any of its traits. This is possibly due to the complex nature of salinity in the effect on leaf area production and the vegetative growth tolerance in, as the osmotic and salt-specific effects of soil salinity are difficult to distinguish (Munns 2002).

The QTL that mapped to chromosome 2AL in the durum landrace, Nax1, accounts for only about 40% of phenotypic variation in leaf Na⁺ concentrations of the F₂ progeny of a cross between Tamaroi and Line 149. Genetic analysis of the distribution of Na⁺ concentration in the progeny of crosses between Line 149 and two parents with high Na⁺ (Tamaroi and Line 141) showed there were two genes of major effect (FIG. 5).

Further evidence for the effect of another gene in addition to the Nax1 QTL was evident from the observation that lines that were heterozygous at the Xgwm312 locus linked to Nax1 occurred in both high and low Na⁺ phenotypes. While lines that were homozygous for the gwm312B allele inherited from the parental source Line 149 were consistently associated with the low Na⁺ phenotype, the heterozygous state was inadequate to identify plants with similar low Na⁺. To account for the low Na⁺ phenotypes found in heterozygous gwm312 plants, a second gene or genomic region independent of Nax1 may provide the full expression of the Na⁺ exclusion trait. As control of Na⁺ transport to the shoots may have multiple sites of operation such as the root cortex, the root stele, and the cells lining the xylem in the upper roots and lower shoot (Munns et al. 2002), and involves a number of membrane transport proteins (Tester and Davenport 2003), the second gene or genomic region may be associated with a different tissue in the plant, or a different set of transport proteins. Despite having some understanding of the physiological basis of the trait (Husain 2002; James et al. 2002), it is still uncertain as to which mechanism is associated with the Na⁺ exclusion locus. The mechanism in tetraploid wheat may be different from that in hexaploid wheat, in particular from the mechanism controlled by the Kna1 locus, as it leads to lower accumulation of Na⁺ at higher external salt concentrations than bread wheat (Husain 2002), and is associated with retention of Na⁺ in the base of the shoots (James et al. 2002). Hence the locus has been designated Nax1.

This study indicates that marker-assisted selection using gwm312 or wmc170 is an effective means of identifying Na⁺ excluding individuals without the need for a phenotypic screen. The robust markers developed have proven useful for the selection of the Na⁺ exclusion trait in durum wheat populations from a range of genetic backgrounds.

Although the Nax1 QTL accounts for approximately 40% of the phenotypic variation, and may denote just one of the two major genes, the markers for this locus can be useful even in the absence of a marker for the second gene. The gwm312B allele is a reliable indicator of low Na⁺. It does not ensure selection of all individuals with the low Na⁺ trait, as some low Na⁺ individuals were heterozygous, selection of the homozygous B allele enriches back-crossed derived lines with the low Na⁺ phenotype.

### Example 3

### Use of the Xpwm312 Marker to Identify Wheat Accessions with the Nax1 Locus

As described above, the wheat accession Line 149 had the Xgwm312 marker associated with the low sodium accumulation phenotype. Further wheat accessions including some durum plants were tested for the presence of the Nax1 locus using the PCR method described above with the Xgwm312 primers. Durum Line 150 (Australian Winter Cereals Collection, Tamworth, NSW, Australia—Accession No. AUS#17050), which showed an intermediate sodium accumulation phenotype in some phenotyping tests, and Line 151 (Australian Winter Cereals Collection, Tamworth, NSW, Australia—Accession No. AUS#17051) which was a low sodium accumulator, together with the three Triticum monococcum lines (C68-101 (Australian Winter Cereals Collection, Tamworth, NSW, Australia—Accession No. AUS# 90382), C68-124 (Australian Winter Cereals Collection, Tamworth, NSW, Australia—Accession No. AUS# 90393), C68-125 (Australian Winter Cereals Collection, Tamworth, NSW, Australia—Accession No. AUS# 90394)) which were parents of durum lines 149, 150 and 151, showed the presence of the Xgwm312 marker. The durum parent of Line 151, Glossy Huguenot was negative for the marker.

In conclusion, it appeared that at least part of the low sodium accumulation phenotype in durum lines 149, 150 and 151 was conferred by the Nax1 gene linked with the Xgwm312 marker and that this gene had originated from the T. monococcum parents of these durum lines.

### Example 4

### Use of Xgwm312 Marker in Hexaploid Wheat and Development of Improved Marker

As described above, the Xgwm312 marker was used as a closely linked marker to distinguish alleles conferring high and low Na⁺ exclusion trait in durum (tetraploid) wheat. In initial experiments, this marker was used to test for the presence of Na⁺exclusion alleles in hexaploid wheat cultivars, for example Camm, Chara, Carnamah and Westonia. PCR reactions using total leaf DNA from plants of these cultivars and the primers Xgwm312R (5′-ACATGCATGCCTACCTAATGG-3′) (SEQ ID NO: 12) and Xgwm312F (5′-ATCGCATGATGCACGTAGAG-3′) (SEQ ID NO: 11) were carried out using standard PCR protocols with Qiagen Hotstar enzyme and buffer, and temperature cycles of 95° C., 15 min for 1 cycle, followed by 94° C., 30 sec; 58° C., 30 sec; 72° C., 2 min for 35 cycles. PCR products were electrophoresed on 2% agarose gels. The main products were fragments of approximately 200 basepairs. In contrast to the clear distinction of alleles in durum wheat (Line 149 vs Tamaroi), the PCR fragments from the hexaploid wheat alleles were only slightly separated and harder to distinguish using standard agarose electrophoresis.

An alternative pair of primers was developed to allow greater distinction between the alleles in hexaploid wheat and durum wheat, as follows. Leaf DNA was extracted from the parental lines Westonia and Line 149, the marker amplified using the primers Xgwm312R and Xgwm312F, and the resultant DNA fragments ligated into pGEM-T Easy vector, followed by transformation into E. coli strain DH5a. The nucleotide sequences of the inserts in three independent clones were determined using plasmid DNA extracted from transformed colonies (FIG. 10). The PCR fragment size from Westonia was 193 basepairs (bp) and from Line 149 187 bp, a difference of 6 bp.

Based on the sequence differences, a new reverse primer was designed: 5′-GTGGGGGAGGCCGGCCAC-3′ (Xgwm312Rmod) (SEQ ID NO: 15) which in reactions with Xgwm312F as the forward primer amplified a 173 bp fragment from Westonia and a 159 bp fragment from Line 149 improving the size difference from 6 bp to 14 bp which is more easily resolved with standard agarose gels. PCR conditions were the same as described above. Thus, a more suitable marker was obtained that produced a clearer separation of bands associated with both alleles, providing a robust co-dominant marker for hexaploid wheat in addition to durum wheat.

### Example 5

### Crossing of Sodium-Exclusion Alleles into Hexaploid Wheat

The sodium exclusion allele on the 2A chromosome of durum wheat, from landrace Line 149, was introduced into representative hexaploid wheat varieties by conventional backcrossing. The hexaploid varieties were chosen as representative of the genetic backgrounds of bread wheats currently grown across the Australian wheat belt. Bread wheats have lower Na⁺ uptake than durum wheats and generally have superior salt tolerance. However, there is about a 2-fold variation in Na⁺ uptake in varieties of bread wheat, which was also represented in the varieties used. Moreover, it is thought that one of the genes controlling the retention of Na⁺ in the leaf sheath is lacking in bread wheat. This may be particularly important where salinity is associated with waterlogging or any soil abiotic/biotic stress that impairs root function. Therefore, it was desirable to introduce sodium exclusion alleles into hexaploid wheat varieties.

Initial crosses were made between the hexaploid cultivars Chara, Camm, Carnamah and Westonia and the tetraploid line P01819 (CSIRO Plant Industry seed catalogue no. P01819) which was a backcrossed (BC₁F₃) selection having a low Na⁺ phenotype identical to that of Line 149 and presumed to contain both Na⁺ exclusion genes. F₁ pentaploids from one of these direct crosses (P02901) were backcrossed with Westonia (male) to produce BC₁F₁ plants, and the progeny then selfed.

The BC₁F₂ seedlings were screened for the presence of the Na⁺ exclusion allele from the tetraploid parent using the Xgwm312 marker. Initially, the Xgwm312 primers as described above were used in the PCR reactions together with analysis on high percentage agarose gels with long run times. Under these conditions, subtle shifts in band position of the PCR products were observed, allowing us to distinguish homozygous material with a reasonable degree of certainty. Later, more robust analyses including those in the Westonia background were performed using the Xgwm31 mod primers as described above.

Three positive lines were identified and selected from two BC₁F₂ populations (P03980 & P03982). These were selfed to produce BC₁F₃ seed. Ears of all 13 selections were determined not to be pentaploid due to full ear fertility and hexaploid status. This was confirmed using a D-genome specific marker (D-gas). These hexaploid BC₁F₃ selections containing the tetraploid Na⁺ exclusion allele were backcrossed again into Westonia and also top-crossed with the hexaploid cultivars Sunstate, Carnamah and Chara. Further backcross/top-crosses were completed without selection using BC₂F₁ plants, and additional top crosses performed into the hexaploid cultivars Janz and Yitpi. BC₃F₂ populations of these crosses were screened using the Xgwm312 marker and selections made, thus generating BC₃F₃ homozygous families containing the tetraploid Na⁺ exclusion allele in 6 different hexaploid backgrounds.

The homozygous lines are tested in both greenhouse and field trials under saline and non-saline conditions for Na accumulation in leaf and grain, growth rate, biomass accumulation and grain yield. For example, the lines are grown in salt-affected fields. Na⁺ uptake in cultivars is substantially decreased in the presence of the Na exclusion alleles from the tetraploid parent, and associated with improved salt tolerance and yield.

### Example 6

### Detection of Sodium Exclusion Trait by Grain Sodium Levels

Grain sodium levels were measured by atomic absorption spectrophotometry for grain from both low sodium (Line 149) and high sodium lines, to test whether the trait could be detected in grain. The plants had been grown in the presence of 1, 75 or 150 mM NaCl (Table 6). The data tabulated below shows that plants having the Na⁺ exclusion trait had low sodium levels in the grain. Grain sodium analysis was therefore a sensitive indicator of the presence of sodium excluding genes, working even when plants were watered with tap water.

TABLE 6
Effect of different levels of salinity on Na⁺
concentration in grain and glumes of Low Na⁺ (Line 149)
and High Na⁺ (Line 141) landraces at grain
maturity. Data show means ± s.e.

Grain Na⁺

| Plant | NaCl level | (μmol g⁻¹ | |
|---|---|---|---|
| phenotype | (mM) | DW) | (mg/kg) |
| Low Na⁺ | 1 | 0.6 ± 0.0 | 14 |
| High Na⁺ | | 1.5 ± 0.2 | 35 |
| Low Na⁺ | 75 | 1.5 ± 0.3 | 35 |
| High Na⁺ | | 9.0 ± 1.0 | 207 |
| Low Na⁺ | 150 | 1.9 ± 0.5 | 44 |
| High Na⁺ | | 17.0 ± 3.6 | 391 |

### Example 7

### Further Genetic and Physical Mapping of the Nax1 Gene

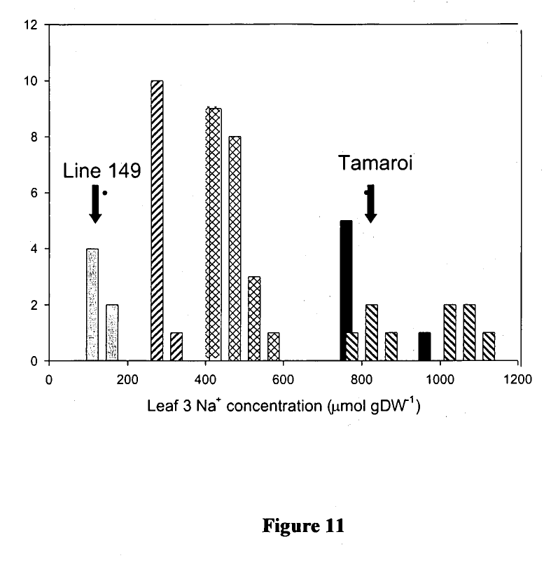

A BC₅F₂ mapping population of 41 phenotyped F₂ lines was generated from a backcross between a BC₄F₂ plant (Example 1) having a low salt accumulation phenotype (Nax1) and Tamaroi, followed by selfing of the BC₅ plants. The source of the gene controlling the low salt (high Na⁺ exclusion) trait in the population was therefore Line 149 and, of the high salt trait, Tamaroi. The 41 lines were phenotyped by growing F2 plants in a glasshouse in gravel culture using the automatic sub-irrigation system as described in Example 1. The level of Na⁺ accumulation in the blade according to Munns et al. (2000). The results are shown in FIG. 11.

The population appeared to be segregating in a 1:2:1 ratio (11:21:9 plants) for the low, medium and high salt subpopulations, indicating the presence of a single co-dominant gene. For genotyping of the lines, plants were grown in soil for about 4 weeks and genomic DNA extracted according to the method of Lagudah and Appels (1991). PCR amplifications for microsatellite and other markers were performed in 20 μL reactions containing 1.5 mM MgCl₂, 2 μM of each primer, 200 μM deoxynucleotide tri-phosphates (dNTPs), 200 μM 1×PCR buffer, 2 units Taq DNA polymerase and 100 ng genomic DNA. Primer sequences for flanking microsatellite markers Xgwm312 were as described in Table 7 and for Xgwm817 as supplied by the supplier

TABLE 7

Markers based on wheat ESTs for mapping
Nax1 on chromosome 2AL

| | | | Restriction |
|---|---|---|---|
| Markers | Designation | Primers (forward and reverse) | enzyme used |
| BF474590 | TaP | (SEQ ID NO: 17) | NcoI |
| | | GGTTAAACCTGGTGCAAATACCC | |
| | | and | |
| | | (SEQ ID NO: 18) | |
| | | TGGAGGCATCCCTCTATAATCAC | |
| BE471258 | TaQ | (SEQ ID NO: 19) | EcoRV |
| | | TTCTGGCATCTCTTCTTCTGGTG | |
| | | and | |
| | | (SEQ ID NO: 20) | |
| | | TATGTCACCCTGATCAAGCACAG | |
| BG262791 | TaA | (SEQ ID NO: 21) | EcoRV |
| | | TGTGGTGCATCACAGGGCTGTTC | |
| | | and | |
| | | (SEQ ID NO: 22) | |
| | | AGCGCTTGCATACTCGTCCGG | |
| BE403217 | TaC | (SEQ ID NO: 23) | NcoI |
| | | AGCAATGAGGATGGTGCTTTCTC | |
| | | and | |
| | | (SEQ ID NO: 24) | |
| | | TGTGAGCGACTCCTCGATTTCAG | |
| Xgwm312 | | (SEQ ID NO: 11) | — |
| | | ATCGCATGATGCACGTAGAG | |
| | | and | |
| | | (SEQ ID NO: 12) | |
| | | ACATGCATGCCTACCTAATGG | |

(TraitGenetics, Gatersleben, Germany). The PCR program and gel electrophoresis conditions were used as described by Lindsay et al. (2004). DNA hybridisations were carried out under stringent conditions using probes labelled with ³²P.

When the population of 41 lines was screened with the Xgwm312 polymorphic microsatellite marker, only one recombinant was observed between the marker and the Nax1 gene, indicating linkage of the marker and the gene. To identify further markers that might be linked even more closely to Nax1, additional microsatellite markers that were thought to map to the 2AL chromosome were tested for polymorphism between the parental lines. Those that showed polymorphism were then tested on the BC₅F₂ population. One marker (Xgwm817) was identified which cosegregated with Nax1 without any recombinants in the population of 41 lines. Based on the map location of Xgwm817, it was predicted that this marker was on the centromere-proximal side of Nax1. Therefore, it was predicted that Xgwm312 and Xgwm817 flanked the Nax1 gene.

To identify further molecular markers linked to Nax1, wheat EST libraries (http://www.ncbi.nlm.nih.gov) were examined for sequences that were homologous (greater than 80% identity at the nucleotide level) to rice genes present in a region on rice chromosome 4L which is syntenic to the region of wheat chromosome 2AL thought to contain Nax1. Twenty-six wheat ESTs were used to identify polymorphisms between Line 149 and Tamaroi. The wheat ESTs were amplified from wheat (cv. Chinese Spring) and durum (Line 149) germplasm, the amplification products cloned into the vector pGEM T-easy and the inserts sequenced to confirm that the correct gene fragment had been isolated. RFLP polymorphisms in Line 149 and Tamaroi corresponding to the wheat ESTs were identified by Southern blot hybridisations using the wheat EST inserts as probes.

Eleven polymorphic ESTs were identified including wheat ESTs with Accession Nos. BF474590, BE471258, BG262791 and BE403217 (designated TaP, TaQ, TaA, TaC, respectively). These had homology to rice genes on BAC Nos. OSJNBb0103I08, OSJNBa0017B10, OSJNBa0013K16 and OSJNBa0058K23, designated OsP, OsQ, OsA, OsC, respectively. These rice genes were contained in the region of rice chromosome 4L, positioned at approximately 27 to 31 Mb. Using the identified polymorphisms (Table 7), the markers TaP, TaQ, TaA and TaC were genetically mapped in the population of 41 BC₅F₂ lines described above. TaP and TaQ were separated by 6 and 5 recombinants (approx 7 cM and 6 cM, respectively) from Nax1 in the BC₅F₂ family and mapped to the centromere-proximal side of Nax1 (FIG. 12). The markers TaA and TaC co-segregated with Xgwm312 and were separated from Nax1 by one recombinant in the BC₅F₂ mapping family, and therefore TaC and TaA were located approximately 1 cM from Nax1 on the centromere-distal side (FIG. 12). The map position of Nax1 was therefore located further away from the centromere than shown in FIG. 6. The discrepancy was probably due to inaccurate reports of the map position of Xpsr102 or XksuD22 on which FIG. 6 was based.

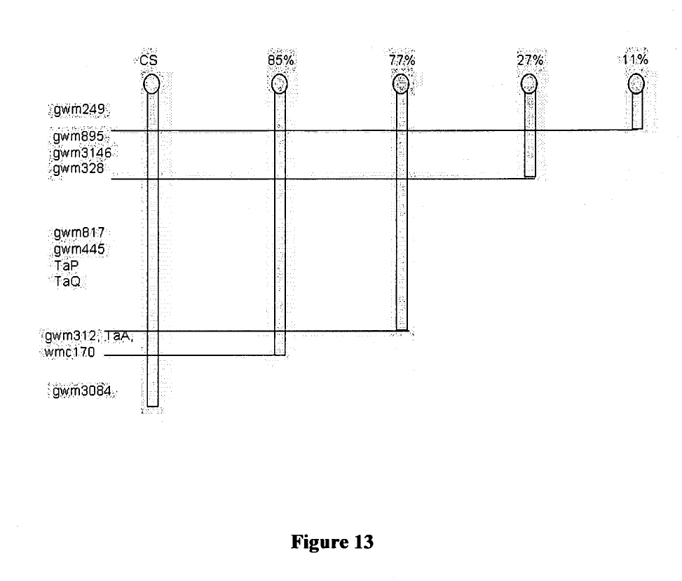

To determine an approximate physical map location of these markers and therefore Nax1, a series of deletion lines in Chinese Spring wheat (Endo and Gill 1996) were used. The wheat ESTs were used as probes in Southern blot hybridisations on DNA from the Chinese Spring deletion lines that had been digested with restriction enzymes (Table 7) that produced a polymorphic RFLP pattern. RFLP marker bands corresponding to the ESTs were thereby located to the physical region delineated by breakpoints 0.27 (CS 2AL-04) representing a telomeric deletion line that retains approx 27% of the 2AL chromosome arm and 0.85 (CS 2AL-1) representing a telomeric deletion line that retains approx 85% of the 2AL chromosome arm (FIG. 13). We therefore predicted that the Nax1 locus was located within a physical segment of the wheat 2AL chromosome between positions 0.27 and 0.85 (with position “0” at the centromere and “1.0” at the tip of 2AL), representing approximately 58% of the chromosome arm.

Further fine mapping of Nax1 will use a population of 12000 BC₅F₂ seeds from the backcross between Line 149 and Tamaroi as described above. DNA isolated from part of each seed will be examined with the markers Xgwm312 and Xgwm817 as described above to identify recombinants between these two markers. The plant corresponding to each recombinant will be recovered from the remainder of each seed and tested for its Nax1 phenotype. Such recombinants will closely define the map position of Nax1.

### Example 8

### Identification of Further Polymorphisms for Use as Molecular Markers

A Line 149 genomic library is made in a λ vector and plaques obtained by plating 106 or more λ infectious particles at high density in a suitable E. coli host. A similar library is made from a wheat plant lacking, for example, the Nax1 allele of interest of Line 149, such as Tamaroi (durum wheat) or Westonia (hexaploid wheat). The plaques are transferred to nylon filters. A Nax1 linked specific probe, such as the PCR fragment amplified from these plants (see, for example, FIG. 10), is labelled with radioactive label and used to hybridise to the nylon filters. Plaques corresponding to spots of hybridisation are isolated and confirmed to be positive for Nax1 linked sequences by second or third rounds of hybridisation. DNA sequencing of the Nax1 gene segments in the λ clones is carried out by standard methods to determine the nucleotide sequence of the surrounding regions amplified by the gwn321 primers. The genomic sequences are compared and further molecular techniques for distinguishing between a Nax1 allele which confers reduced sodium accumulation, as well as enhanced tolerance to saline and/or sodic soils, and a Nax1 allele which does not confer this phenotype can be devised.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Asch, F. et al. (2000) Euphytica 113:109-118.
Dubcovsky, J. et al. (1996) Theoretical and Applied Genetics 2:448-454.
Dvo\check{r}ák, J. et al. (1994) Theoretical and Applied Genetics 87:872-877.
Eagles, H. A. et al. (2001) Australian Journal of Agricultural Research 52:1349-1356.
Ellis, R. P. et al. (1997) New Phytologist 137:149-157.
Ellis, R. P. et al. (2002) Journal of Experimental Botany 53:1163-1176.
Endo, T. R. and Gill, B. S. (1996). Journal of Hereditary 87:295-307.
Francois, L. E. et al. (1986) Agronomy Journal 78:1053-1058.
Garcia, A. et al. (1995) Theoretical and Applied Genetics 90:1106-1111.
Gorham, J. et al. (1987) Theoretical and Applied Genetics 74:584-548.
Gorham, J. et al. (1990) Planta 180:590-597
Gorham, J. et al. (1997). New Phytologist 137:109-116.
Harker, N. et al. (2001) Australian Journal of Agricultural Research 52:1121-1130.
Husain, S. (2002) A thesis submitted for the degree of Doctor of Philosophy of the Australian National University, Canberra, Australia.
Husain, S. et al. (2003) Australian Journal of Agricultural Research 54:589-597.
James, R. A. et al. (2002) ComBio2002, Sydney: Combined Conference Abstracts.
Kingsbury, R. W. and Epstein, E. (1984) Crop Science 24:310-314.
Koomeef, M. and Stam, P. (2001) Plant Physiology 125:156-159.
Lagudah, E. S. and Appels, R. (1991) Genome 34:387-395.
Langridge, P. et al. (2001) Australian Journal of Agricultural Research 52:1043-1077.
Lemieux, B. (2000) Current Genomics. 1: 301-311.
Lindsay, M. et al. (2004) Functional Plant Biology 31:1105-1114.
Maas, E. V. and Grieve, C. M. (1990) Crop Science 30:1309-1313.
Manly, K. F. et al. (2001) Mammalian genome 12:930-932.
Martin, P. K. et al. (1994) Aspects of Applied Biology 39:215-222.
Miller, D. M. (1987) Plant Physiology 85:164-166.
Munns, R. et al. (1995) Australian Journal of plant Physiology 22:561-569.
Munns, R. et al. (2000) Australian Journal of Agricultural Research 51:69-74.
Munns, R. (2002) Plant, Cell and Environment 25:239-250.
Munns, R. et al. (2002) Plant and Soil 247:93-105.
Munns, R. and James, R. A. (2003) Plant and Soil 253:201-218.
Munns, R. et al. (2003) Australian Journal of Agricultural Research 54:627-635.
Nachit, M. M. et al. (2001) Theoretical and Applied Genetics 102:177-186.
Nicolas, M. E. et al. (1993) Australian Journal of Plant Physiology 20:349-360.
Pitman, M. G. and Läuchli, A. (2002) In ‘Salinity: environment-plants-molecules’. (Ed. A. Lauchli and U. Luttge) pp. 3-20. (Kluwer Academic: Dordrecht, The Netherlands).
Rawson, H. M. et al. (1988) Australian Journal of Agricultural Research 39:759-772.
Rengasamy, P. (2002) Australian Journal of Experimental Agriculture 42:351-361.
Rivelli, A. R. et al. (2002) Functional Plant Biology 29:1065-1074.
Roder, M. S. et al. (1998) Genetics 149:2007-2023.
Rogers, M. E. and Noble, C. L. (1992) Plant and Soil 146:131-136.
Sayed, H. I. (1985) Theoretical and Applied Genetics 69:651-657.
Seah, S. et al. (1998) Theoretical and Applied Genetics 97:937-945
Shah, S. H. et al. (1987) Journal of Experimental Botany 38:254-269.
Sharp P. J. et al. (2001) Australian Journal of Agricultural Research 52:1357-1366.
Tester, M. and Davenport, R. (2003) Annals of Botany 91:503-527.
Vos, P. et al. (1995) Nucleic Acids Research 23:4407-4414.
Weimberg, R. (1987) Physiol Pant. 70:381-388.

1. A method of identifying a wheat plant having a phenotype of enhanced tolerance to saline and/or sodic soils, and/or reduced sodium accumulation in an aerial part of the plant, the method comprising detecting a nucleic acid molecule of the plant, wherein the nucleic acid molecule is linked to a Nax1 locus of wheat that comprises an allele that confers on the plant the phenotype of enhanced tolerance to saline and/or sodic soils and/or reduced sodium accumulation in an aerial part of the plant.

2. (canceled)

3. The method of claim 1 which comprises:
i) hybridising a second nucleic acid molecule to said nucleic acid molecule which is obtained from said plant,
ii) optionally hybridising at least one other nucleic acid molecule to said nucleic acid molecule which is obtained from said plant; and
iii) detecting a product of said hybridising step(s) or the absence of a product from said hybridising step(s).

4. The method of claim 3, wherein the second nucleic acid molecule is used as a primer to reverse transcribe or replicate at least a portion of the nucleic acid molecule.

5. The method of claim 1, wherein the nucleic acid is detected using a technique selected from the group consisting of: restriction fragment length polymorphism analysis, amplification fragment length polymorphism analysis, microsatellite amplification and/or nucleic acid sequencing.

6. The method of claim 1 which comprises nucleic acid amplification.

7. (canceled)

8. The method of claim 6, wherein the amplification is performed using primers which amplify a polymorphic GA-repeat, wherein the polymorphic GA-repeat can be amplified using the primers ATCGCATGATGCACGTAGAG (SEQ ID NO: 11) and ACATGCATGCCTACCTAATGG (SEQ ID NO: 12).

9. The method of claim 6, wherein the amplification is 5 performed using primers which amplify a polymorphic GA-repeat, wherein the polymorphic GA-repeat can be amplified using the primers ATCGCATGATGCACGTAGAG (SEQ ID NO: 11) and GTGGGGGAGGCCGGCCAC (SEQ ID NO: 15).

10. The method of claim 6, wherein the amplification is performed using a primer comprising the sequence ATCGCATGATGCACGTAGAG (SEQ ID NO: 11), in conjunction with a primer comprising the sequence ACATGCATGCCTACCTAATGG (SEQ ID NO: 12) or GTGGGGGAGGCCGGCCAC (SEQ ID NO: 15), or at least one primer which is a variant of any one of said primers.

11. The method of claim 6, wherein the amplification is performed using primers which amplify a polymorphic repeat, wherein the polymorphic repeat can be amplified using the primer pairs selected from:
i) ACATCCACGTTTATGTTGTTG (SEQ ID NO: 13) and TTGGTTGCTCAACGTTTACTT (SEQ ID NO: 14),
ii) TGTGGTGCATCACAGGGCTGTTC (SEQ ID NO:21) and AGCGCTTGCATACTCGTCCGG (SEQ ID NO:22), and
iii) AGCAATGAGGATGGTGCTTTCTC (SEQ ID NO:23) and TGTGAGCGACTCCTCGATTTCAG (SEQ ID NO:24).

12. The method of claim 6, wherein the amplification is performed using primers pairs comprising the sequences selected from:
i) ACATCCACGTTTATGTTGTTG (SEQ ID NO: 13) and TTGGTTGCTCAACGTTTACTT (SEQ ID NO: 14), or at least one primer which is a variant of any one of said primers,
ii) TGTGGTGCATCACAGGGCTGTTC (SEQ ID NO:21) and AGCGCTTGCATACTCGTCCGG (SEQ ID NO:22), or at least one primer which is a variant of any one of said primers, and
iii) AGCAATGAGGATGGTGCTTTCTC (SEQ ID NO:23) and TGTGAGCGACTCCTCGATTTCAG (SEQ ID NO:24) or at least one primer which is a variant of any one of said primers.

13. A method of selecting a wheat plant from a population of wheat plants, the method comprising;
i) crossing two wheat plants of which at least one plant comprises a Nax1 locus comprising an allele which confers on the plant a phenotype of enhanced tolerance to saline and/or sodic soils, and/or reduced sodium accumulation in an aerial part of the plant, and
ii) screening progeny plants from the cross for the presence or absence of said Nax1 locus by a method of claim 1,
wherein progeny with said allele have a phenotype of enhanced tolerance to saline and/or sodic soils, and/or reduced sodium accumulation in an aerial part of the plant, when compared to progeny lacking said allele.

14-15. (canceled)

16. The method of claim 13, wherein the wheat is tetraploid wheat, preferably durum wheat, or at least one of the wheat plants of step i) is a hexaploid wheat plant.

17-18. (canceled)

19. The method of claim 13, wherein the cross is between a durum wheat plant comprising said allele and a hexaploid wheat plant lacking said allele.

20. The method of claim 13, wherein one of the wheat plants is Line 149, Line 150, Line 151 or a progenitor or progeny plant thereof comprising said allele.

21. A method of introducing a Nax1 locus into the genome of a wheat plant lacking said locus, the method comprising;
i) crossing a first parent wheat plant with a second parent wheat plant, wherein the second plant comprises a Nax1 locus which comprises an allele which confers on the second plant a phenotype of enhanced tolerance to saline and/or sodic soils and/or reduced sodium accumulation in an aerial part of the plant, and
ii) backcrossing the progeny of the cross of step i) with plants of the same genotype as the first parent plant for a sufficient number of times to produce a plant with a majority of the genotype of the first parent but comprising said allele,
wherein progeny plants are screened for the presence or absence of said allele by a method of claim 1.

22. (canceled)

23. The method of claim 21, wherein the first and/or second parent wheat plant is a durum wheat plant, or the first parent wheat plant is a hexaploid wheat plant.

24. (canceled)

25. The method of claim 21, wherein one of the wheat plants is Line 149, Line 150, Line 151 or a progenitor or progeny plant thereof comprising said allele.

26-27. (canceled)

28. A wheat plant comprising an allele of the Nax1 gene on chromosome 2AL which confers on the wheat plant a phenotype of enhanced tolerance to saline and/or sodic soils, and/or reduced sodium accumulation in an aerial part of the plant.

29. A seed of a wheat plant of claim 28.

30. A product produced from a wheat plant of claim 28 or a seed of claim 29, wherein the product comprises genetic material comprising an allele of the Nax1 gene on chromosome 2AL which confers on the wheat plant a phenotype of enhanced tolerance to saline and/or sodic soils, and/or reduced sodium accumulation in an aerial part of the plant.

31. (canceled)

32. The product of claim 30, wherein the product is a food product.

33-39. (canceled)


SEQUENCE LISTING (24 sequences)

SEQ ID NO: 1 - DNA, 177 bp, Triticum turgidum ssp. durum cv. Line 149
tgtgcacgtagagtagatgtgatgtagggctcgtcgttacttgggtggtggagtgagtgtgaagccgttatgatttttgaccactgccccgtgcccgcgggaaaacaaaggcggggagagagagagagagaggtggccggcctcccccacccccaccattaggtaggcatgcatgta

SEQ ID NO: 2 - DNA, 184 bp, Triticum aestivum cv. Westonia
tgagcacgtagagtagatgtgatgtagggctcgtcgttacttgggtggtggagtgagtgtgaagccattatgatttttgaccactgccccgtgcccgcgggaaaacaaaggcggggagagagagagagagagagagagagagagaggtggccggcctcccccaccattaggtaggcatgcatgt

SEQ ID NO: 3 - DNA, 16 bp, Artificial Sequence
  Oligonucleotide primer
gacgatgagtcctgag

SEQ ID NO: 4 - DNA, 14 bp, Artificial Sequence
  Oligonucleotide primer
tactcaggactcat

SEQ ID NO: 5 - DNA, 17 bp, Artificial Sequence
  Oligonucleotide primer
gatgagtcctgagtaag

SEQ ID NO: 6 - DNA, 18 bp, Artificial Sequence
  Oligonucleotide primer
cacgatggatccagtgca

SEQ ID NO: 7 - DNA, 17 bp, Artificial Sequence
  Oligonucleotide primer
gacgtgctacctaggtc

SEQ ID NO: 8 - DNA, 18 bp, Artificial Sequence
  Oligonucleotide primer
gatggatccagtgcagag

SEQ ID NO: 9 - DNA, 20 bp, Artificial Sequence
  Oligonucleotide primer
caaatggatcgagaaaggga

SEQ ID NO: 10 - DNA, 22 bp, Artificial Sequence
  Oligonucleotide primer
ctgccatttttctggatctacc

SEQ ID NO: 11 - DNA, 20 bp, Artificial Sequence
  Oligonucleotide primer
atcgcatgatgcacgtagag

SEQ ID NO: 12 - DNA, 21 bp, Artificial Sequence
  Oligonucleotide primer
acatgcatgcctacctaatgg

SEQ ID NO: 13 - DNA, 21 bp, Artificial Sequence
  Oligonucleotide primer
acatccacgtttatgttgttg

SEQ ID NO: 14 - DNA, 21 bp, Artificial Sequence
  Oligonucleotide primer
ttggttgctcaacgtttactt

SEQ ID NO: 15 - DNA, 18 bp, Artificial Sequence
  Oligonucleotide primer
gtgggggaggccggccac

SEQ ID NO: 16 - DNA, 18 bp, Artificial Sequence
  Oligonucleotide primer
tagatggtgatgtagggc

SEQ ID NO: 17 - DNA, 23 bp, Artificial Sequence
  Oligonucleotide primer
ggttaaacctggtgcaaataccc

SEQ ID NO: 18 - DNA, 23 bp, Artificial Sequence
  Oligonucleotide primer
tggaggcatccctctataatcac

SEQ ID NO: 19 - DNA, 23 bp, Artificial Sequence
  Oligonucleotide primer
ttctggcatctcttcttctggtg

SEQ ID NO: 20 - DNA, 23 bp, Artificial Sequence
  Oligonucleotide primer
tatgtcaccctgatcaagcacag

SEQ ID NO: 21 - DNA, 23 bp, Artificial Sequence
  Oligonucleotide primer
tgtggtgcatcacagggctgttc

SEQ ID NO: 22 - DNA, 21 bp, Artificial Sequence
  Oligonucleotide primer
agcgcttgcatactcgtccgg

SEQ ID NO: 23 - DNA, 23 bp, Artificial Sequence
  Oligonucleotide primer
agcaatgaggatggtgctttctc

SEQ ID NO: 24 - DNA, 23 bp, Artificial Sequence
  Oligonucleotide primer
tgtgagcgactcctcgatttcag